(19) United States
(12) Patent Application Publication
(10) Pub. No.: US 2012/0129248 A1
(43) Pub. Date: May 24, 2012

(54) ASSAY TOOLS AND METHODS OF USE

(75) Inventors: Mark S. Chee, Encinitas, CA (US); Igor A. Kozlov, San Diego, CA (US)

(73) Assignee: PROGNOSYS BIOSCIENCES, INC., La Jolla, CA (US)

(21) Appl. No.: 13/388,229

(22) PCT Filed: Aug. 2, 2010
(86) PCT No.: PCT/US2010/044134

§ 371 Date: Jan. 31, 2012

Related U.S. Application Data
(60) Provisional application No. 61/230,583, filed on Jul. 31, 2009.

(51) Int. Cl. G01N 33/53; C12M 1/40

(52) U.S. Cl. 435/287.1; 422/69

(57) ABSTRACT

The present invention provides assay tools for the detection of biological or chemical activity in a sample. The assay tools of the invention provide direct detection using a positive signal generated on a surface of the assay tool. These assay tools provide improved methods for detection and/or identification of multiple agents (e.g., enzymes) in a sample, analysis of substrate specificity of such agents, and binding affinities and specificities of such agents. Upon activity a component is released from a first immobilised construct and then captured by a capture surface. At least two different immobilised constructs are used.

### FIELD OF THE INVENTION

This invention relates to assay tools for detection of biological and/or chemical activity in a sample.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Robust methods to analyze the genome and transcriptome have been developed, and these methods have opened a new frontier in the use of genetics and gene expression for a myriad of uses, including diagnostics, prognostics, and understanding of evolutionary trends. Although these tools are quite powerful, the information they provide is limited, as many proteins are regulated later in the protein biosynthesis pathways, e.g., through post-translational modifications. Such post-translational modifications include attachment to other biochemical functional groups such as acetate, phosphate, various lipids and carbohydrates, by changing the chemical nature of an amino acid (e.g., citrullination) or by making structural changes, like the formation of disulfide bridges. Also, enzymes may remove amino acids from the amino end of the protein, or cleave the peptide chain to provide an active fragment of the originally translated protein. Thus, typical gene expression analysis, which measures the presence or level of a particular gene or transcript, is often not sufficient information to indicate the level of cellular activity of a protein. Consequently, functional analysis of the proteome is a frontier of major importance.

Enzymes are a class of proteins that are generally activated by post-translational activities. For example, proteases are nearly exclusively regulated by posttranslational modifications (Turk, Nat Rev Drug Discov. 2006 September; 5(9):785-99). The majority of proteases are synthesized as zymogens and are activated only in specific subcellular compartments or upon stimulation. Furthermore, many proteases have endogenous inhibitors that attenuate their destructive capacity (Overall C M and Blobel C P, Nat Rev Mol Cell Biol. 2007 March; 8(3):245-57. Epub 2007 Feb. 14). Since monitoring of protease activity can be used in diagnosis and prognosis of disease (Hang H C and Ploegh H, Chem. Biol. 2004 October; 11(10):1328-30; López-Otín C and Overall C M, Nat Rev Mol Cell Biol. 2002 July; 3(7):509-19), direct assays of protease activity are needed to fully understand the role of proteases, e.g., in normal and disease states.

Assays for various enzymatic activities have been developed, but many have limitations and they are generally not well suited for high throughput screening of complex biological mixtures. For example, assays for individual proteases are well established, and there are many commercial kits for single protease activity assays, including those designed for compound library screening and lead identification of protease drug candidates. Although single protease assays are relatively straightforward, they are expensive and limited in the amount of information that they can provide per unit of sample.

Assays for multiplex detection of protease activity, such as those provided in U.S. Pat. No. 7,229,769, require multistep processes and transfer of reagents between steps. This increases both the cost of the assay procedure and the risk of error within and between steps.

There is thus a need for a cost-effective, sensitive multiplex assay for the identification of enzyme activity in a biological sample. The present invention addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The invention provides tools and methods for the detection of a chemical or biological cleavage event and/or competitive binding of a molecule. The tools of the invention enable detection of the cleavage and/or competitive binding event using a positive signal generated on a surface of the assay tool. In one general aspect, these tools provide improved methods for detection and/or identification of multiple different cleavage agents (e.g., chemicals, restriction endonucleases or proteases) in a sample. In another general aspect, the tools can be used to identify substrate selectivity and/or suitability using one or a few active cleavage agents and multiple substrates. In yet other general aspects, by using the tools for competitive binding assays, binding moieties that have a desired affinity for particular binding regions can be identified.

In one aspect the invention provides an assay tool for detecting biological or chemical activity in a sample, where the tool comprises: a set of at least two different immobilized constructs comprising a releasable component; and a capture surface for binding of the releasable components upon a displacement event. Upon a given displacement event, a releasable component is released from the immobilized construct and interacts with the capture surface to enable the generation of a detectable signal on the capture surface. The detectable signal can be generated directly, e.g., through excitation of a fluorescent moiety that is a part of the releasable component, or indirectly, e.g., through binding of a detectable marker to the releasable component on the capture surface.

In specific aspects, the capture surface comprises one or more capture agents for selective binding of the releasable component. In this aspect, the releasable components preferably comprise an affinity region that selectively binds to a capture agent on the capture surface. In other specific aspects, the displacement event is an enzymatic cleavage event.

In another aspect, the invention provides an assay tool for detecting biological or chemical activity in a sample, where the tool comprises a set of at least two different immobilized constructs having a cleavage agent substrate region, an affinity region, and a detectable marker, and a capture surface. The affinity region and the detectable marker of a construct are released from the construct upon induced cleavage of the construct, and binding of a released affinity region and detectable marker on a capture surface enables generation of a positive signal on the capture surface.

In some aspects, the immobilized constructs of the set comprise a substrate region for the same cleavage agent. In other aspects, the immobilized constructs of the set comprise substrate regions for different cleavage agents. In yet other aspects, two or more immobilized constructs of the set comprise different detectable markers. In yet other aspects, the detectable markers of the two or more constructs of the set are substantially identical.

In certain aspects, the capture surfaces for use in the invention comprise one or more capture agents. In preferred aspects, the capture agents selectively bind to an affinity region on a releasable component of an immobilized construct. In specific aspects, the capture agents of the set are substantially identical, and the constructs can comprise substantially the same affinity region. In other aspects, the capture surface comprises two or more different capture agents that detect different affinity regions.

In specific aspects, the invention provides a tool for detecting cleavage agent activity in a sample comprising a set of at least two different immobilized constructs having a cleavage agent substrate region, an affinity region, and a detectable marker; and a set of capture agents wherein a detectable positive signal is generated as a result of binding of the released detectable marker. The affinity region and the detectable marker are released from the construct upon induced cleavage of the constructs and bind to a corresponding capture agent. This tool can have constructs with the same cleavage agent substrate regions or substrates for different cleavage agents.

In other specific aspects, the invention provides a tool for detecting cleavage agent activity in a sample comprising a surface having 1) a set of two or more immobilized constructs comprising a cleavage agent substrate, an affinity region and a detectable marker, and 2) a set of immobilized capture agents that generate a detectable positive signal resulting from cleavage of the substrate and binding of a released detectable marker. The released affinity region and detectable marker bind selectively to a corresponding capture agent on the surface, producing a positive signal at the site of the capture agent.

In yet other aspects, the invention provides a tool for detecting cleavage agent activity in a sample comprising: 1) a set of two or more constructs comprising a cleavage agent substrate, an affinity region and a detectable marker immobilized to a first surface; and 2) a set of capture agents immobilized to a second surface. The affinity region and the detectable marker are released from the construct upon induced cleavage and bind selectively to a corresponding capture agent. The first and second surfaces are in a proximity that allows detection of a positive signal generated as a result of binding of an affinity region and a detectable marker to a specific capture agent on the second surface. In yet other aspects, the invention provides a tool for detecting cleavage agent activity in a sample comprising a surface having a set of two or more immobilized constructs, the constructs comprising 1) an affinity region associated with a first component of a combined detectable marker, 2) a capture agent associated with a second component of a combined detectable marker, and 3) a substrate for cleavage by a cleavage agent between the affinity region and the capture agent. Cleavage of the substrate results in release of the affinity region. The released affinity region binds to the capture agent following cleavage of the substrate, generating a positive signal as a result of an interaction of the first and second components of the combined detectable marker.

In a specific aspect, the present invention provides tools and methods for the detection of enzymatic activity in a sample, e.g., a biological sample. The tools of the invention enable detection using a positive signal generated on a surface of the assay tool. These tools provide improved methods for detection and/or identification of multiple enzymes (e.g., restriction endonucleases or proteases) in a sample or alternatively for identification of enzyme selectivity and/or substrate suitability using one or a few enzymes and multiple substrates. In certain aspects, the substrate constructs have a protease cleavage site. In other aspects, the substrate constructs have a nucleic acid cleavage site.

In certain aspects, the invention provides a tool for detecting enzyme activity in a sample comprising a set of at least two different immobilized constructs having an enzyme substrate region, an affinity region, and a detectable marker; and a set of capture agents that generate a detectable positive signal as a result of binding of the released detectable marker. The affinity region and the detectable marker are released from the surface upon enzyme-induced cleavage of the constructs and bind selectively to a corresponding capture agent. This tool can have constructs with the same enzyme substrate regions or substrates for different enzymes. In certain aspects, the substrate constructs have a protease cleavage site. In other aspects, the substrate constructs have a nucleic acid cleavage site.

In another aspect, the invention provides a tool for detecting enzyme activity in a sample comprising a surface with 1) a set of two or more immobilized constructs comprising an enzyme substrate, an affinity region, and a detectable marker, and 2) a set of immobilized capture agents that generate a detectable positive signal as a result of binding of a detectable marker immobilized to the same surface. The affinity region and the detectable marker are released from the surface upon enzyme-induced cleavage of the constructs and bind selectively to a corresponding capture agent on the same surface. In specific aspects, the capture agent comprises a nucleic acid, and the detectable marker is associated with a nucleic acid complementary to the capture agent. In other specific aspects, the capture agent comprises a peptide and the detectable marker is associated with a peptide that specifically binds to the capture agent. The proximity of the set of constructs to corresponding detectable moieties allows detection of the binding of a detectable marker to a specific capture agent.

In yet other aspects, the invention provides an assay tool for detecting activity of two or more enzymes in a sample comprising a set of two or more immobilized constructs having an enzyme substrate, an affinity region and a detectable marker immobilized to a first surface and a set of immobilized capture agents that generate a detectable positive signal as a result of binding of a detectable marker immobilized to a second surface. The first and second surfaces are positioned in the tool in a proximity that allows binding of a detectable marker to a specific capture agent on the second surface. In specific aspects, the capture agent comprises a nucleic acid and the detectable marker is associated with a nucleic acid complementary to the capture agent. In other specific aspects, the capture agent comprises a peptide and the detectable marker is associated with a peptide that specifically binds to the capture agent. The proximity of the set of constructs to corresponding detectable moieties allows detection of the binding of a detectable marker to a specific capture agent.

In a specific aspect, the invention provides an assay tool for detecting competitive binding of a series of molecules, comprising a surface having a set of two or more immobilized constructs comprising an affinity region, an agent bound to the affinity region; and a set of immobilized capture agents in proximity to the constructs; wherein a detectable positive signal is generated as a result of competitive binding and release of the bound agent from the affinity region. In some aspects, the bound agent is directly labeled with a detectable marker. In other aspects, the bound agent is detected following displacement from the construct and binding to the capture agent.

In another specific aspect, the invention provides an assay tool for detecting competitive binding of a molecule in a sample, comprising 1) a set of two or more constructs comprising an affinity region, and an agent comprising a detectable marker bound to the affinity region immobilized to a first surface, and 2) a set of capture agents immobilized to a second surface. The first and second surfaces are in a proximity that enables detection of a positive signal created through competitive binding and release of the agent from the affinity region, and a detectable positive signal is generated as a result of competitive binding and release of the bound agent from the affinity region. In some aspects, the bound agent is directly labeled with a detectable marker. In other aspects, the bound agent is detected following displacement from the construct and binding to the capture agent.

In certain aspects, competitive binding refers to the displacement of an agent by a molecule with higher affinity to substantially the same binding site as the molecule of interest, i.e. replacement of the bound agent with the new agent on substantially the same site on the affinity region. In other aspects, competitive binding refers to allosteric binding, i.e. binding at a second location on the affinity region or other part of the construct, that causes a release of the agent via a conformational change of the construct.

In one aspect, the invention provides a method for detecting a modulator of enzyme activity, comprising providing an assay tool comprising a set of two or more constructs comprising a cleavage agent substrate, an affinity region and a detectable marker immobilized to a first surface and a set of capture agents immobilized to a second surface. The first surface is exposed to an enzyme and a modulator or putative modulator of the enzyme. Modulation of the enzyme activity can be detected due to the presence or absence of positive signals on the second surface. In a preferred aspect, the method further comprises comparing the positive signals created by the enzyme in the is presence of the modulator to the positive signal created by the enzyme on the same cleavage agent substrate in the absence of the modulator or in the presence of different concentration of the modulator, and the activity is determined through comparison of signals generated in the presence or absence of the modulator, or in the presence of various concentrations of the modulator.

In yet another specific aspect, the invention provides an assay tool for detecting inhibitors of enzymatic activity, comprising a set of two or more immobilized constructs having an enzyme substrate, an affinity region and a detectable marker immobilized to a first surface and a set of immobilized capture agents that generate a detectable positive signal as a result of binding of a detectable marker immobilized to a second surface. The assay is carried out utilizing a set of enzymes that can cause a displacement event of a releasable construct of one or more immobilized constructs. The enzyme can be introduced to the immobilized constructs in the presence and/or absence of an enzyme inhibitor or a putative enzyme inhibitor to identify the extent to which different enzymes are inhibited under certain assay conditions. A use would be in drug development, where instead of screening vs one protease at a time, one could screen against many potential targets, e.g. to look for off-target effects.

The capture agent used to identify the cleavage and/or competitive binding activity can be any member of a reactive pair that interacts with the other member of the reactive pair in the assay tool of the invention. In some aspects, the capture agent comprises a nucleic acid, and the detectable marker is associated with a labeled nucleic acid complementary to the capture agent. In other aspects, the capture agent comprises a peptide, and the detectable marker is associated with a peptide that specifically binds to the capture agent.

In specific aspects, the capture agent comprises a nucleic acid, and the detectable marker is associated with a nucleic acid complementary to the capture agent. In other specific aspects, the capture agent comprises a peptide and the detectable marker is associated with a peptide that specifically binds to the capture agent. The proximity of the set of constructs to corresponding detectable moieties allows detection of the binding of a detectable marker to a specific capture agent.

In the tools of the invention, the assay comprises defined regions of two or more substantially identical constructs. In a specific example, the surface comprises two or more identical constructs that are separated by a physical and/or chemical barrier. In a particular embodiment, the identical constructs are separated into channels on the surface.

In addition to the described tools, the invention also provides methods for detecting the activity of a cleavage agent in a sample. Such methods can be used to determine the presence of different cleavage agents in a complex sample, the substrate specificity of one or a few such cleavage agents, the effect of different assay conditions on cleavage of substrates, and the like.

In one aspect, the invention provides a method for detecting cleavage agent activity in a sample, comprising: 1) providing a surface having i) a set of two or more immobilized constructs comprising a cleavage agent substrate, an affinity region and a detectable marker, and ii) a set of immobilized capture agents that generate a detectable positive signal as a result of binding of a detectable marker; 2) exposing the surface to a sample under conditions that allow cleavage agents in the sample to act on the constructs, and 3) detection of one or more positive signals on the surface generated by the activity of the cleavage agents on the constructs in the sample. A detected positive signal is indicative of the activity of a cleavage agent in the sample.

In another aspect, the invention provides a method for detecting cleavage agent activity in a sample, comprising: 1) providing an assay tool comprising i) a set of two or more constructs comprising a cleavage agent substrate, an affinity region and a detectable marker immobilized to a first surface; and ii) a set of capture agents immobilized to a second surface; wherein the first and second surfaces are in a proximity that enables detection of a positive signal generated as a result of binding of a detectable marker to a specific capture agent on the second surface; 2) exposing the surface to a sample under conditions that allow cleavage agents in the sample to act on the constructs, and 3) directly detecting one or more positive signals on the surface generated as a result of activity of cleavage agents present in the sample on the immobilized constructs. A detected positive signal is indicative of the activity of a cleavage agent in the sample.

In yet another aspect, the invention provides methods for detecting competitive binding of an agent in a sample. The competitive binding may be indicative of a higher affinity interaction at substantially the same binding site, allosteric interaction with a construct that results in the release of a bound agent, and the like.

In one aspect, the invention provides a method for detecting competitive binding in a sample, comprising: 1) providing a surface comprising i) a set of two or more immobilized constructs comprising an affinity region, and an agent comprising a detectable marker associated with the affinity region; and ii) a set of immobilized capture agents in proximity to the constructs; wherein a detectable positive signal is generated upon competitive binding and release of the agent from the affinity region 2) exposing the surface to a sample under conditions that allow agents in the sample to bind to the constructs, and 3) detecting one or more positive signals on the surface generated as a result of the competitive binding of the agents in the sample. A detected positive signal is indicative of the competitive binding activity of an agent in the sample.

In another aspect, the invention provides a method for detecting competitive binding in a sample, comprising: 1) providing an assay tool comprising a set of two or more constructs comprising an affinity region, and an agent comprising a detectable marker associated with the affinity region immobilized to a first surface, and a set of capture agents immobilized to a second surface; wherein the first and second surfaces are in a proximity that allows detection of a positive signal generated as a result of competitive binding and release of the agent from the affinity region; 2) exposing the surface to a sample under conditions that allow agents in the sample to bind to the constructs, and detecting one or more positive signals on the surface generated as a result of the competitive binding of the agents in the sample. A detected positive signal is indicative of the competitive binding activity of an agent in the sample.

The invention also provides methods for detecting enzyme activity in a sample, comprising providing a surface having 1) a set of two or more immobilized constructs comprising an enzyme substrate, an affinity region and a detectable marker, and 2) a set of immobilized capture agents that generate a detectable positive signal as a result of binding of a detectable marker; exposing the surface to a sample under conditions that allow enzymes in the sample to act on the constructs; and detecting one or more positive signals on the surface created by the activity of the enzymes on the constructs in the sample; wherein a detected positive signal is indicative of the activity of an enzyme in the sample.

The invention further provides methods for detecting enzyme activity in a sample, comprising providing a first surface having a set of two or more immobilized constructs comprising an enzyme substrate, an affinity region and a detectable marker; providing a second surface in proximity to the first surface, where the second surface comprises a set of immobilized capture agents which produce a detectable positive signal upon binding of the released detectable marker from the first surface; exposing at least the first surface to a sample under conditions that allow enzymes in the sample to act on the constructs, and directly detecting one or more positive signals on the second surface created by the activity of the enzymes on the constructs in the sample.

In specific aspects of the invention, the constructs of the invention are cleavable by a single protease. In other aspects, the constructs of the invention are cleavable by a selected subset of proteases, e.g., two or more members of a protease class or two or more proteases with defined activity. In yet other aspects, the constructs are cleavable by novel activity of a protease or by proteases not known to exist.

The positive signal detected on the surface of the assay tool is preferably generated using a single detectable marker. In certain aspects, however, the positive signal can be generated using a combined detectable marker that is created upon binding of the cleaved substrate to the capture agent. In yet other aspects, the positive signal can be generated through higher order (2°, 3°, etc.) labeling schemes (e.g. secondary antibody labeling.)

In specific aspects, subsets of constructs are physically separated on the surface, e.g., by a physical and/or chemical barrier. This allows different subset areas to be treated individually, or different test samples to be introduced to different parts of the surface.

### BRIEF DESCRIPTION OF THE DRAWINGS

FIG. 1 illustrates a first general scheme for a two-surface tool of the invention.

FIG. 2 illustrates a specific aspect of a tool of FIG. 1 having a universal detection surface, a single detectable marker, and constructs comprising different cleavage agent substrates.

FIG. 3 illustrates a specific aspect of a tool of FIG. 1 having a universal detection surface, different detectable markers, and constructs comprising different cleavage agent substrates.

FIG. 4 illustrates a specific aspect of a tool of FIG. 1 having two or more capture agents and affinity regions, a single detectable marker, and constructs comprising different cleavage agent substrates.

FIG. 5 illustrates a specific aspect of a tool of FIG. 1 having two or more capture agents and affinity regions, different detectable markers, and constructs comprising different cleavage agent substrates.

FIG. 6 illustrates a specific aspect of a tool of FIG. 1 having constructs and capture agents separated by a physical barrier, e.g., a spacer unit.

FIG. 7 illustrates a general scheme for a single surface tool of the invention comprising a construct and a capture agent immobilized to the same surface.

FIG. 8 illustrates one general scheme for the single surface tool of FIG. 7 comprising detection of the detectable moiety using a combined detectable marker.

FIG. 9 illustrates another general scheme for the single surface tool of FIG. 7 comprising detection of the detectable moiety using a combined detection agent, where the construct serves as both the affinity region and the capture agent.

FIG. 10 illustrates a specific aspect of a single surface tool of FIG. 7 comprising a construct having a peptide cleavage site, an oligonucleotide capture agent, and a single detectable marker.

FIG. 11 illustrates a specific aspect of a single surface tool of FIG. 7 comprising a construct having a nucleic acid cleavage site, an oligonucleotide capture agent, and a single detectable marker.

FIG. 12 illustrates a specific aspect of a single surface tool of FIG. 7 comprising a construct having a peptide cleavage site, a capture agent, and a single detection agent.

FIG. 13 illustrates a first aspect of a double surface tool that measures competitive binding of two or more molecules based on the binding affinity to a construct.

FIG. 14 illustrates a second aspect of a double surface tool that measures competitive binding of two or more molecules based on the binding affinity to a construct.

FIG. 15 illustrates a third aspect of a double surface tool that measures competitive binding of two or more molecules based on the binding affinity to a construct.

FIG. 16 illustrates a specific aspect of the single surface tool in which the surface is patterned, e.g., to create areas that are depressed with respect to the planar surface.

FIG. 17 illustrates a second specific aspect of the single surface tool in which the surface is patterned, e.g., to create areas that are depressed with respect to the planar surface, and wherein the constructs are located in the depressed regions.

FIG. 18 illustrates a second specific aspect of the single surface tool in which the surface is patterned, e.g., to create areas that are depressed with respect to the planar surface, and wherein the constructs and the capture agents are found in the depressed regions.

FIG. 19 illustrates a specific aspect of the single surface tool comprising beads with immobilized constructs on a surface.

FIG. 20 illustrates exemplary schemes for a single surface assay of the invention comprising multiple construct features and/or multiple reporter regions on the surface of the invention.

FIG. 21 is a schematic of a cartridge for use in constructing the assay tools of the invention.

FIG. 22 is a photo illustrating fluorescent detection on a surface comprising both thrombin protease substrates and TEV substrates using either a thrombin protease or a TEV protease.

FIG. 23 is a bar graph illustrating signal achieved versus background for the protease assay using two proteases and two substrates.

FIG. 24 is a bar graph illustrating levels of fluorescent detection of cleavage on a surface compared to background.

FIG. 25 is a photo illustrating fluorescent detection on a surface comprising two substrates using only a thrombin protease.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “binding pair” means any two molecules that are known to selectively bind to one another. In the case of two proteins, the molecules selectively bind to one another with a high affinity as described in more detail herein. Examples include, but are not limited to, specific interactions such as biotin and avidin; biotin and streptavidin; an antibody and its particular epitope; and the like. Examples also include non-specific interactions including but not limited to hydrophobic-hydrophobic, electrostatic, molecular (van der Waals); and the like. The term also includes complementary nucleic acid molecules that selectively hybridize at or above a desired melting temperature.

The term “complementary” refers to the topological compatibility or interactive structure of interacting surfaces of a nucleic acid binding pair. Preferred complementary structures have binding affinity for each other and the greater the degree of complementarity the nucleic acids have for each other the greater the hybridization between the structures.

The term “diagnostic tool” as used herein refers to any composition or assay of the invention used in order to carry out a diagnostic test or assay on a patient sample. As a diagnostic tool, the composition of the invention may be considered a collection of analyte specific reagents, and as such may form part of a diagnostic test regulated by a federal or state agency. The use of the compositions of the invention as a diagnostic tool is not intended to be related to any use of the composition in the development of therapeutic agents.

The term “displacement event” refers to any event that results in the release of a releasable component from an immobilized construct, e.g., an enzymatic event such as a cleavage event caused by a protease, a competitive binding of a molecule that results in the release of a releasable component, and the like. Examples include but are not limited to degradation (e.g., thermal and chemical (i.e. pH)), dissociation (e.g., electrostatic induced and nucleic acid duplex melting), fragmentation (e.g. high energy, such as in mass spectrometry) and digestion (e.g. multiple enzymes.)

The term “enzyme-induced cleavage” includes any enzymatic activity that directly or indirectly leads to the cleavage of a substrate. The term as used herein includes direct cleavage of a substrate by an enzyme, e.g., cleavage of a peptide substrate by a protease or cleavage of a nucleic acid substrate by a restriction endonuclease, as well as indirect cleavage of an enzyme, e.g. the binding of the enzyme to the substrate allows binding of a cofactor that induces cleavage or the enzyme binds to another cofactor that induces the cleavage. Enzyme-induced cleavage also includes enzymatic modification of a substrate, e.g., dephosphorylation by a phosphatase that renders the substrate susceptible to cleavage by another enzyme.

The term “induced cleavage” includes any activity of a cleavage agent that directly or indirectly leads to the cleavage of a substrate. The term as used herein includes direct cleavage of a substrate by a chemical moiety, a protein, a ribozyme, or is other similar naturally-occurring, synthetic or recombinant molecule. Induced cleavage also includes physical interactions including but not limited to photo-induced cleavage.

“Nucleic acid” and “oligonucleotide” are used herein to mean a polymer of nucleotide monomers. As used herein, the terms may refer to single stranded or double stranded forms. Monomers making up nucleic acids and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like, to form duplex or triplex forms. Such monomers and their internucleosidic linkages may be naturally occurring or may be analogs thereof, e.g., naturally occurring or non-naturally occurring analogs. Non-naturally occurring analogs may include peptide nucleic acids, locked nucleic acids, phosphorothioate internucleosidic linkages, bases containing linking groups permitting the attachment of labels, such as fluorophores, or haptens, and the like. Whenever the use of an oligonucleotide or nucleic acid requires enzymatic processing, such as extension by a polymerase, ligation by a ligase, or the like, one of ordinary skill would understand that oligonucleotides or nucleic acids in those instances would not contain certain analogs of internucleosidic linkages, sugar moieties, or bases at any or some positions, when such analogs are incompatible with enzymatic reactions. Nucleic acids typically range in size from a few monomeric units, e.g., 5-40, when they are usually referred to as “oligonucleotides,” to several hundred thousand or more monomeric units. Whenever a nucleic acid or oligonucleotide is represented by a sequence of letters (upper or lower case), such as “ATGCCTG,” it will be understood that the nucleotides are in 5′.fwdarw.3′ order from left to right and that “A” denotes deoxyadenosine, “C” denotes deoxycytidine, “G” denotes deoxyguanosine, and “T” denotes deoxythymidine, “I” denotes deoxyinosine, “U” denotes uridine, unless otherwise indicated or obvious from context. Usually nucleic acids comprise the natural nucleosides (e.g., deoxyadenosine, deoxycytidine, deoxyguanosine, deoxythymidine for DNA or their ribose counterparts for RNA) linked by phosphodiester linkages; however, they may also comprise non-natural nucleotide analogs, e.g., modified bases, sugars, or internucleosidic linkages. To those skilled in the art, where an enzyme has specific oligonucleotide or nucleic acid substrate requirements for activity, e.g., single-stranded DNA, RNA/DNA duplex, or the like, then selection of appropriate composition for the oligonucleotide or nucleic acid substrates is well within the knowledge of one of ordinary skill, especially with guidance from treatises, such as Sambrook et al., Molecular Cloning, Second Edition (Cold Spring Harbor Laboratory, New York, 1989), and like references.

The terms “peptide”, “polypeptide,” and the like are used interchangeably herein, and refer to a polymeric form of amino acids of any length, which can include coded and non-coded amino acids, chemically or biochemically modified or derivatized amino acids, and polypeptides having modified peptide backbones.

The term “positive signal” means any generated signal associated with a releasable component that is bound to the capture surface, i.e. a “gain of signal” resulting from the interaction between a cleavage product and a capture surface. This includes the generation of a direct positive signal on the capture surface, e.g., through the capture of a cleavage product that itself generates a detectable signal upon binding to the capture surface (e.g., chemiluminescence) or through interaction between the affinity region and the capture agent (e.g., FRET detection) or indirectly, through the generation of a positive signal from a capture agent that binds to the cleavage product following an interaction between the cleavage product and the capture surface, e.g., through the use of a labeled antibody that selectively binds to the cleavage product. A detectable positive signal may include, but is not limited to, an increase in fluorescence, chemiluminescence, radioactivity, or any other agent easily detected using conventional techniques.

The term “reactive pair” as used herein refers to any two molecules that can interact to indicate the binding of a capture agent to a construct cleavage product or other molecule that has been displaced from a construct, e.g., a molecule that is displaced from the construct due to competitive binding of a molecule from a sample. Reactive pairs include binding pairs as well as other interactive molecules, such as those that cause a chemical reaction resulting in creation of a covalent bond.

The term “releasable component” refers to the portion of an immobilized construct that is released upon a displacement event.

The term “research tool” as used herein refers to any composition or assay of the invention used for scientific enquiry, academic or commercial in nature, including the development of pharmaceutical and/or biological therapeutics. The research tools of the invention are not intended to be therapeutic or to be subject to regulatory approval; rather, the research tools of the invention are intended to facilitate research and aid in such development activities, including any activities performed with the intention to produce information to support a regulatory submission.

The term “selectively binds”, “selective binding” and the like as used herein, when referring to a binding partner (e.g., protein, nucleic acid, antibody, etc.), refers to a binding reaction of two or more binding partners that result in the generation of a statistically significant positive signal under the designated assay conditions. Typically the interaction will subsequently result in a detectable signal that is at least twice the standard deviation of any signal generated as a result of undesired interactions (background).

The term proximity is used to define the spatial relationship between the construct and binding moiety such that the cleavage product is capable of interacting with the binding moiety either through diffusion, fluidic flow and/or any means of transport not prohibited by the physical nature of the assay.

The term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation, Tₘ=81.5+0.41 (% G+C), when a nucleic acid is in aqueous solution at 1M NaCl (see e.g., Anderson and Young, Quantitative Filter Hybridization, in Nucleic Acid Hybridization (1985). Other references (e.g., Allawi, H. T. & SantaLucia, J., Jr., Biochemistry 36, 10581-94 (1997)) include alternative methods of computation which take structural and environmental, as well as sequence characteristics into account for the calculation of Tₘ.

### DETAILED DESCRIPTION OF THE INVENTION

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; and Berg et al. (2002) Biochemistry, 5^{th} Ed., W.H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a substrate” refers to one or more copies of a substrate, and reference to “the assay” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

The Invention in General

The present invention provides powerful methods for measuring displacement of a releasable portion of an immobilized peptide following a reaction event with one or more enzymes, and in particular with enzymes involved in nucleic acid or protein cleavage. The present invention provides detection of a positive signal (i.e., “gain-of-signal” detection) to identify and/or detect activity of one or more specific enzymes in a sample. A specific example of this is the release of a cleavage portion (i.e., the releasable component), of an immobilized substrate comprising a cleavage site for cleavage activity and an affinity region that binds to a capture surface. Upon dissociation of the releasable portion (e.g., the cleavage portion) of the immobilized construct, the releasable portion is captured by binding to the capture surface. In specific aspects, the affinity region of the releasable portion selectively binds to a capture agent that is associated with the capture surface. Thus, in certain aspects the invention provides assays for high-throughput analysis of cleavage agent activity by creating detectable cleavage products that generate a positive signal as a result of binding to corresponding capture agents.

Although other methods are available for detecting enzymatic activity, the present invention has many characteristics that offer a significant improvement over the current state of the art. For example, the assays and methods of the present invention allow not only detection of multiple enzymes of one class in a sample (e.g., identification and/or detection of multiple restriction endonucleases or proteases), but also allows the user to distinguish the presence or absence of activity of enzymes with different specific activities. The ability to distinguish the activity of different enzymes in a high throughput manner allows a more comprehensive analysis of a sample, such as a biological sample, using a single assay tool.

In a particular aspect the invention provides a high throughput method to detect protease activity in a sample. Currently available tools for identifying protease activity of multiple proteases, such as “universal peptide” arrays, allow detection of protease activity that may be caused by several different proteases, but cannot distinguish which specific proteases are responsible for this activity when there are multiple potential proteases in a sample. Assays that do offer multiplexed protease activity assays, such as those described in U.S. Pat. No. 7,229,769, require multiple steps including a purification step prior to detection of a signal. The direct detection of a gain-of-signal provided by the assay tools of the invention greatly simplifies the assay methodology compared to such multi-step high throughput analysis techniques, reducing time and cost for performing the assay.

Loss-of-signal (“LOS”) assays may be hindered by the excess of immobilized constructs, which diminishes the dynamic range of the assay. The assays of the present invention are not limited by large amounts of constructs, and in fact the assay may gain sensitivity and other benefits from the presence of a large number of constructs. LOS assays may also have background issues due to immobilized inactive constructs, e.g., constructs with poorly synthesized peptides, or unpurified. Such inactive constructs can be a major issue for LOS assays as they would be a constant contribution to the background.

The GOS assays of the present invention do not have this issue of background from products that would not cleave as they would not dissociate and transfer to the capture surface. The present invention is thus a significant improvement over conventional array-based protease activity assays since it is very sensitive, robust, versatile, and cost-effective.

The present invention provides tools that utilize either a single detection agent that is present in the releasable portion of the immobilized construct or detection schemes involving two or more components. Single detection agent aspects are preferred, as they are generally a more cost effective way to detect more than one protease in a sample and do not require complicated synthesis techniques, multiplexing, and/or decoding procedures to identify the enzymatic activity.

In addition to testing activity of multiple enzymes in a sample, the invention provides assay tools for assaying activity of one or more individual enzymes of interest (for example, a single protease) against a large number of peptide substrates on a surface. This will enable the determination of selectivity of individual enzymes or classes of enzymes against varying substrates, and can also aid in identifying optimal substrates for enzymatic activity under specific assay conditions.

In certain aspects, the invention provides an assay tool for detecting inhibitors of enzymatic activity, e.g., general or specific inhibitors of protease activity. Such assays are carried out utilizing a set of enzymes that can cause a displacement event, e.g., through a cleavage event that causes the release of the releasable component of one or more immobilized constructs. For example, a protease or a group of proteases can be introduced to the immobilized constructs in the presence of a protease inhibitor or a putative inhibitor. By comparing the signals generated in the presence vs absence of the inhibitor or putative inhibitor, the specificity and efficiency of inhibition can be determined. This aspect has utility in, for example, drug development, where the assay would facilitate screening of multiple proteases and cleavage substrates against multiple inhibitors or putative inhibitors. In addition to identifying targets, this would aid in providing safety data with respect to potential off-target effects.

In certain aspects, the invention provides an assay tool for detecting an activator of enzymatic activity, including general and/or specific enhancers of enzymatic activity requiring a co-factor or an agent that activates enzymatic activity, e.g., agents that convert a zymogen into the active form of an enzyme. Such assays are carried out utilizing a set of enzymes that can cause a displacement event resulting in the release of the releasable component of one or more immobilized constructs. This aspect has utility in, for example, identifying enhancers of known or potential drugs or for identifying factors that may increase the risk of an unintended side effect of a therapeutic agent.

The assay tools of the invention could thus effectively be used for any agent that modulates enzyme activity. The assay tools of the invention can be used to screen for protein agonists as well as antagonists, and include numerous small molecule, peptide, or other types of activators and inhibitors.

The ability to detect an increase in signal rather than a decrease allows higher sensitivity in the assay system, by greatly reducing any background signal or noise, and thus the ability to identify smaller quantities of enzyme that may be present in a complex sample. It is also advantageous because these assays require a small amount of sample for analysis. This allows large-scale experiments that are too difficult or costly to be practical using current methods. The high-throughput assays will accelerate the discovery of research tools for the study of cellular function and regulation.

The assay tools of the invention also provide tools and methods for identifying and/or optimizing substrates for cleavage or displacement activity. For example, constructs on the tool can represent a library of molecules with varied structures of the cleavable moiety and/or changes in context, and the tools can be used to screen for cleaving activity of an agent in particular conditions, including light, pH, ion concentration, presence of specific reactive species, etc.

In another aspect the tools can be used to screen for competitive binders. In such aspects, for example, displacement of a molecule based on a higher binding affinity to the affinity region of a construct can be used to detect a signal. In another example, allosteric binding at a second location on the affinity region or other part of the construct that causes a release of the agent via a conformational change of the construct can be detected.

Interactions that could be tested for competitive binding include macromolecular interactions (e.g., peptide+peptide; nucleic acid+nucleic acid; target+aptamer etc.) or small molecule interactions (e.g., drug+target, hapten+antibody).

Capture Surfaces for Use in the Invention

The capture surface for use in the present invention is a surface designed to capture one or more released affinity regions to enable the generation of a positive signal. The capture surface for use in the present invention can use various mechanisms and/or capture agents for the capture of the released affinity regions. For example, the capture surface may be modified to be hydrophobic or hydrophilic, such as coating the surface exposed to the released affinity region with a hydrophobic or hydrophilic agent, and the affinity region may be captured directly by virtue of its interaction with the surface. In another example, the capture surface may comprise a substantially uniform coating of a plurality of a single capture agent, such as avidin or streptavidin. In yet another example, the capture surface may comprise specific regions with positioned capture regions, e.g., a surface with a single capture agent in defined capture regions. In certain aspects, the capture surface may comprise adjacent capture regions with different capture agents having specificity for different affinity regions. In other aspects, the capture regions are adjacent to or surrounded by non-functionalized or blocked regions of the capture surface that do not bind to the releasable components. These capture regions may each comprise a single capture agent, or they may comprise two or more capture agents that can selectively bind to two or more released affinity regions.

Surfaces suitable to the assay can be of any geometric shape and/or orientation that enable detection of the cleavage product through binding to a capture surface. In some aspects, surfaces of the invention are functionalized, biocompatible and of a shape and relative orientation that is suited to the volumes and physical properties (diffusion length etc.) of the particular assay. Suitable surfaces include materials such as glass, silicon, quartz, polyacrylamide-coated glass, ceramics, silica, various plastics, and the like. In some aspects, the surface is not functionalized, and the cleavage agent interacts with the capture surface using non-specific binding. In other aspects, the surface is functionalized in one or more regions, or “capture regions.” In other aspects, the capture structure is functionalized on all or substantially all of the capture surface.

The capture surface itself may be, for example, a substantially planar surface, a well or a platform, a particle, bead or microsphere. In one aspect, the surface is a bead. In another aspect, the surface is a planar surface. In yet another aspect, assays that use a multitude of surfaces can be any combination of shapes. Typically, for conventional uses, the planar surface is in the range of from 0.02 to 20 cm² or larger and is determined primarily by the detection methods employed and the ability to resolve (e.g., in the case of fluorescent markers, the ability to optically resolve) the different constructs and/or regions of constructs on the surface.

Immobilized Constructs

The immobilized constructs for use in the present invention comprise a displacement region that allows the release of a releasable component upon introduction of an agent. In specific aspects, the immobilized construct comprises an enzyme substrate region that results in an enzymatic displacement or release of a portion of the construct upon exposure to the appropriate enzyme, and an affinity region for binding to the capture substrate that is in the portion of the construct released upon exposure to the enzyme. In other specific aspects, the displacement region allows release of the releasable component upon binding of an agent that effectively displaces the releasable component from the immobilized constructs. In any of these aspects, these regions may be distinct regions, or there may be overlapping structure between the substrate region and the affinity region, provided the affinity region is sufficiently intact upon release to allow it to bind with sufficient affinity to the capture surface for the purposes of the assay.

In certain aspects, the immobilized construct comprises an attachment region, an enzyme substrate region and a releasable component comprising an affinity region, and a detectable marker. The detectable marker allows detection of the releasable component on the capture surface following release of the releasable component as a result of a displacement event and binding of this component to the capture surface. The affinity region may be distinct from the detectable marker, may overlap in structure with the detectable marker, or in certain aspects the detectable marker itself may be the affinity region that facilitates binding of the releasable component to the capture surface.

The enzyme substrate portion of the constructs can be a peptide, a nucleic acid, a polysaccharide, or a molecule containing other types of enzyme cleavable bonds. Examples of enzymes that can act on peptide and related substrates such as peptidoglycans include proteases, phosphatases, glycohydrolases (e.g., lysozyme), and the like. Examples of nucleic acid substrates include oligonucleotide regions that can be cleaved by a DNA or RNA enzyme, e.g., a restriction endonuclease or the like.

Detection of a signal, following binding of the affinity region of the releasable component to the capture surface, can be read directly from the single surface or, in the case of the dual-surface aspects, after separating the two surfaces, e.g., using a standard microarray scanner, a chemiluminescent detection kit, mass spectrometry, or other conventional means available to those skilled in the art. In certain aspects, however, the detection of the signal from the two surface aspects can be read without separating the two functionalized surfaces. For example, a confocal-type scanner with a short depth of field could be used to collect signal from the surface comprising the capture agents. In another example, evanescent wave illumination could be used to excite molecules on the surface comprising the capture agents. Such methods may offer the advantage of detection of signal in real time or at specific intervals, and may provide information on the kinetics of the reaction of the reactive pair and/or binding pair on the capture surface.

In different tools of the invention, linkers may be used to attach the capture agent and/or the construct to a surface. Numerous types of linkers can be used, and the linker will generally be selected on the type of construct, (amino acid, nucleic acid, etc.), the desired properties of the linker (length, flexibility) and other similar characteristics. Such linkers may comprise nucleotides, polypeptides, or a suitable synthetic material. The linker structures are preferably hydrocarbon base polymers which are comprised of biocompatible polymeric materials (e.g., polyethylene glycol).

In certain aspects, the surface immobilized constructs and/or the capture agents comprise a cleavable linker directly attached to the surface that allows the other components of the construct to be separated from the surface independently from any cleavage event at the site of the enzyme substrate. In some aspects, the cleavable linker will be the same or identical for all of the surface-immobilized constructs. In other aspects, subsets of constructs on the surface will have the same cleavable linker, which differ from the cleavable linker of the other subsets on the same surface.

Detectable Markers

The detectable marker for use with the invention can be any molecule that generates or permits the generation of a positive signal upon the binding of the affinity region to the capture surface. Such detectable markers include, but are not limited to, an antibody, a radioisotope, fluorescent compound, bioluminescent compound, a chemiluminescent compound, a molecular marker, or optical labels, e.g., visible deposits of gold and/or silver nanoparticles. The detectable marker and affinity region can be one in the same. In a preferred aspect, the detectable marker is a fluorescent dye. Suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue™, Texas Red, and others described in the 6th Edition of Molecular Probes Handbook by Richard P. Haugland.

In certain aspects, the detectable marker is a combined detectable marker that comprises two or more molecules that interact with another molecule to create the positive signal. In these aspects, the detectable marker could be a dye that interacts to create a Forster resonance energy transfer (FRET). Suitable FRET dyes include, but are not limited to, coumarin-labeled phospholipids (CC2-DMPE), bis-(1,3-dialkylthiobarbituric acid) trimethine oxonol, and DisBac (see, Gonzalez and Maher, Receptors and Channels 8). Combined detectable markers also include moieties that can be detected via binding of a labeled molecule, e.g. a hapten with its carrier, a ligand that is detectable using an introduced labeled antibody, and the like.

In the case of a nucleic acid, labels can be attached to nucleotides at a variety of locations, and attachment can be made either with or without a linker. Conventionally used nucleotide analogs for labeling of nucleic acid with fluorophores generally have the is fluorescent moiety attached to the base of the nucleotide substrate molecule. It can also be attached to a sugar moiety (e.g., deoxyribose) or the alpha phosphate. See Zhu et al., “Directly Labelled DNA Probes Using Fluorescent Nucleotides with Different Length Linkers,” Nucleic Acids Res. 22: 3418-3422 (1994), which is hereby incorporated by reference.

Assay Tools: Two-Surface Configuration

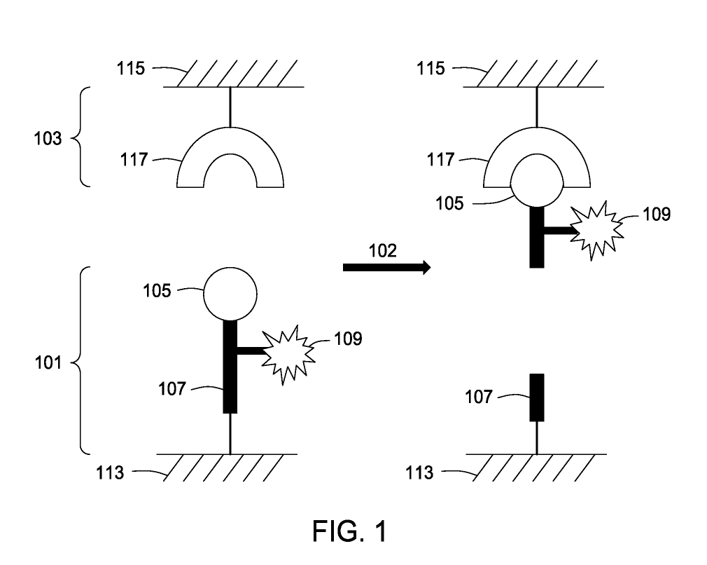

In one aspect of the invention, the assay tools are composed of two surfaces with a known, constant spatial relationship. In certain aspects, the surfaces are solid supports with a known, constant spatial relationship, such as a planar surface, a film, beads or a combination thereof. In a preferred aspect, this aspect of the invention provides two planar surfaces. Different specific examples of such aspects are illustrated in FIGS. 1-4.

The assay tool is constructed of a first surface having a set of immobilized peptide constructs, and a second surface having a set of immobilized capture agents as illustrated in FIG. 1. As shown, the first surface 113 has attached thereto constructs 101 that comprise an enzymatic cleavage agent substrate 107, a detectable marker 109 and an affinity region 105 that binds specifically to a capture agent 117 that is immobilized 103 on the second surface 115 in direct proximity to the construct 101. Prior to cleavage of the enzyme substrate 107 of the construct 101, the affinity region 105 and the capture agent 117 are kept separate by the immobilization to their respective surfaces. Following cleavage of the enzyme substrate 107, the affinity region and the detectable marker are released from the first surface 113, and diffuse to the adjacent capture agent that is found proximally on the second surface 115. In certain aspects of the invention, the affinity region 105 and the capture agent 117 comprise a reactive pair. The reactive pair is bound by a chemical reaction, e.g., a covalent binding, which allows detection of the reactive pair via the associated detectable marker.

In other aspects of the invention, the affinity region 105 and the capture agent 117 comprise a specific protein binding pair. In yet other aspects, the affinity region 105 and the capture agent 117 comprise complementary nucleic acids. Numerous binding pairs may be utilized for such detection, as will be apparent to one skilled in the art upon reading the present disclosure.

This aspect of the invention effectively provides for a transfer of signal from the enzyme substrate to a detection surface, and the positive signal can be achieved through detection of the gain of signal on the capture surface. This transfer is specific to the cleavage of immobilized enzyme substrate, and the cleavage of particular enzyme substrates is indicative of the presence of that enzyme in a sample.

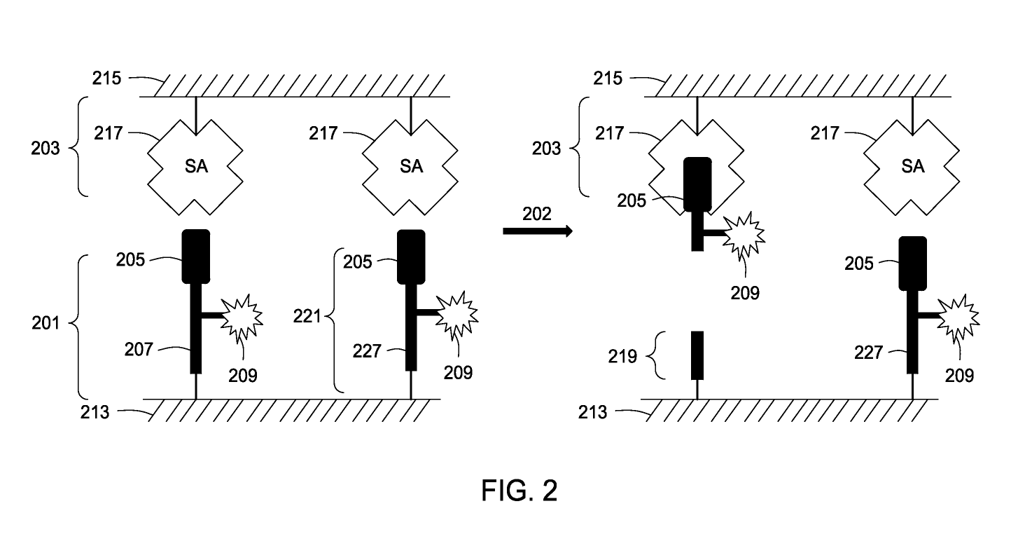

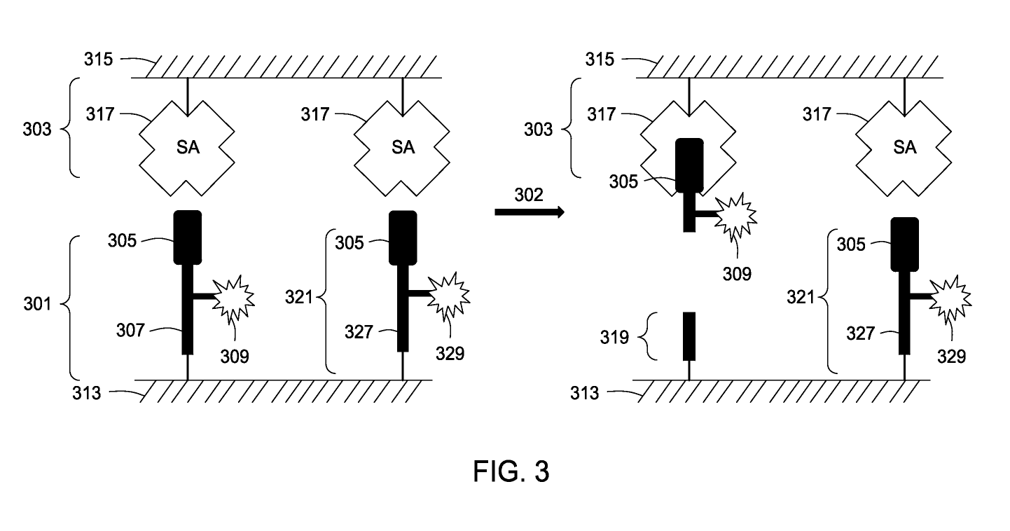

In certain aspects, the assay tool is constructed of a first surface having a set of two or more immobilized constructs with different cleavage agent substrates and a second detection surface comprising two or more copies of the same capture agent. The capture agents on the second surface generally comprise capture constructs that are one component of a binding pair or a reactive pair, and the other half of the pair is located on the affinity region of the construct on the first surface. FIG. 2 and FIG. 3 illustrate specific examples of this aspect. In FIG. 2, a first surface 213 having a set of two or more immobilized constructs 201, 221 both having biotin affinity regions 205 and different cleavage agent substrates 207, 227 is brought into proximity with a second surface 215 having a set of capture agents 203 comprising streptavidin molecules 217 immobilized thereto. Upon cleavage 202 of the substrate 207 with a target cleavage agent, a portion of the construct 219 remains on the first surface while the biotin 205 and detectable marker 209 diffuse to the capture agent 217 on the capture surface 215, where the cleavage event can be detected by the binding of the biotin 205 and the detectable marker to the capture agent 217. In FIG. 2, each of the detectable labels 209 is the same, and the spatial location of the positive signal on the assay tool is used to identify the specific substrate that was cleaved in the assay, and is indicative of the presence of a cleavage agent in the sample tested.

FIG. 3 is similar to FIG. 2, except the different constructs employ different detectable markers. In FIG. 3, a first surface 313 having a set of two or more immobilized constructs 301, 321 both having biotin affinity regions 305 and different cleavage agent substrates 307, 327 is brought into proximity with a second surface 315 having a set of capture agents 303 comprising streptavidin molecules 317 immobilized thereto. Upon cleavage 302 of the substrate 307 with a target cleavage agent, a portion of the construct 319 remains on the first surface while the biotin 305 and detectable marker 309 diffuse to the capture agent 317 on the capture surface 315, where the cleavage event can be detected by the binding of the biotin 305 and the detectable marker to the capture agent 317. In FIG. 3, the detectable labels 309, 329 differ on at least two or more constructs, and the positive signal generated (as well as spatial location in certain aspects) is used to identify the specific substrate that was cleaved in the assay, and is indicative of the presence of a cleavage agent in the sample tested.

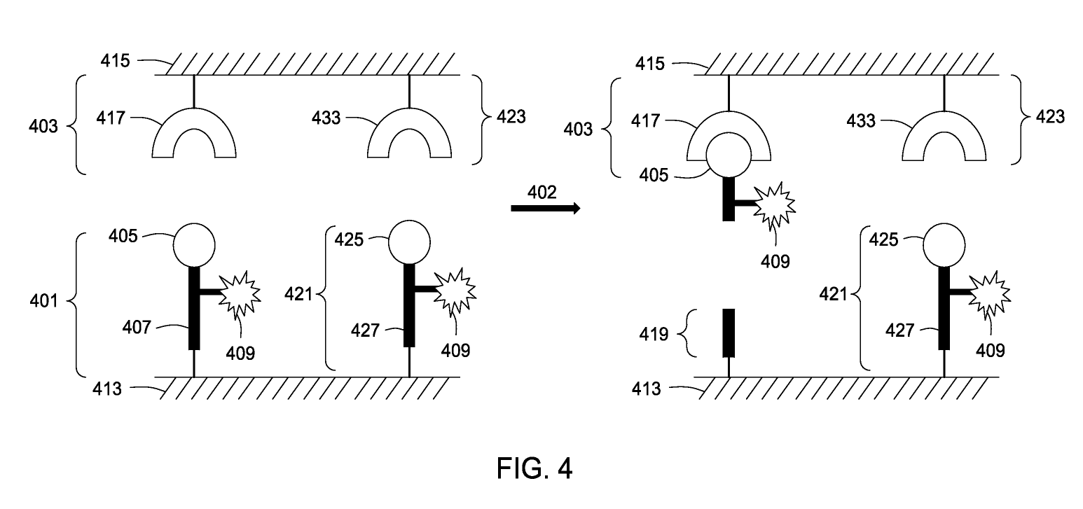

In other aspects of the invention, different capture agents and affinity regions can be used on different constructs to create the reactive pair (e.g., binding pairs) interaction on the second surface. In FIG. 4, the assay tool is constructed of a first surface 413 having a set of two or more immobilized constructs 401, 421, and a second surface 415 having a set of immobilized capture agents 403 and 423, which comprise different capture agents (417 and 433, respectively). The second surface 415 is located in proximity to the first surface 413. As shown, the first surface 413 has immobilized thereto constructs 401, 421 that comprise different cleavage agent substrates 407, 427 and the same detectable marker 409, and different affinity regions 405, 425 that bind specifically to their corresponding capture agents (403, 423 respectively). Upon cleavage 402 of a construct, a portion of the construct 419 remains on the first surface and the cleavage product comprising the affinity region 405 and the detectable marker 409 diffuses and binds to the corresponding capture agent 417 in direct proximity on the second surface 415. The positive signal generated on the second surface and the spatial location of this signal are used to identify the specific substrate that was cleaved in the assay, and is indicative of the presence of a cleavage agent in the sample tested.

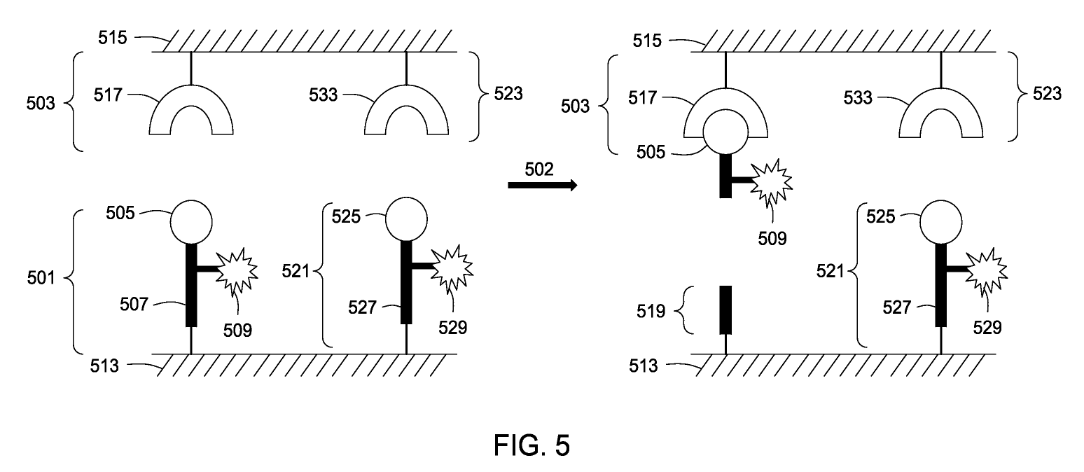

FIG. 5 is similar to FIG. 4, except the different constructs may employ different detectable markers. In other aspects of the invention, different capture agents and affinity regions can be used on different constructs to create the reactive pair (e.g., binding pairs) interaction on the second surface. In FIG. 5, the assay tool is constructed of a first surface 513 having a set of two or more immobilized constructs 501, 521, and a second surface 515 having a set of immobilized capture agents 503 and 523, which comprise different capture agents (517 and 533, respectively). The second surface 515 is located in proximity to the first surface 513. As shown, the first surface 513 has immobilized thereto constructs 501, 521 that comprise different cleavage agent substrates 507, 527, different detectable markers 509, 529 and different affinity regions 505, 525 that bind specifically to their corresponding capture agents (503, 523 respectively). Upon cleavage 502 of a construct, a portion of the construct 519 remains on the first surface and the cleavage product comprising the affinity region 505 and the detectable marker 509 diffuses and binds to the corresponding capture agent 517 in direct proximity on the capture surface 515. The positive signal generated on the second surface (as well as spatial location in certain aspects) is used to identify the specific substrate that was cleaved in the assay, and is indicative of the presence of a cleavage agent in the sample tested.

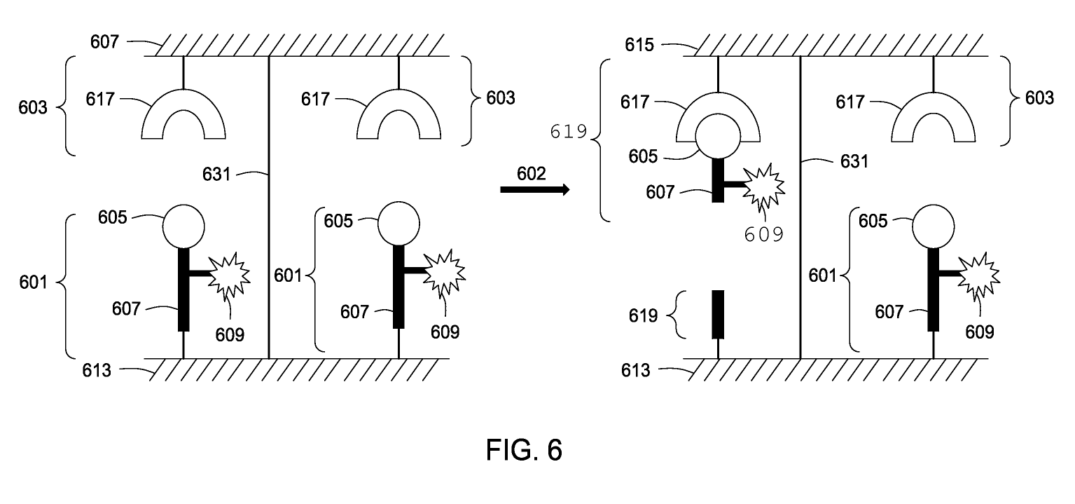

In certain aspects of the invention, it may be desirable to physically separate different constructs and/or capture agents. These may comprise any of the construct/capture agent combinations that are described in the specification. One exemplary tool is illustrated in FIG. 6, in which the assay tool is constructed of a first surface 613 having a set of two or more immobilized constructs 601 that are substantially the same, and a second surface 615 having a set of capture agents 617 immobilized to it is placed in proximity to the first surface 613. As shown, the first surface 613 has attached thereto constructs 601 that comprise cleavage agent substrates 607, detectable markers 609 and affinity regions 605 that bind specifically to their corresponding capture agents 617. A barrier 631 physically separates the different constructs and capture agents. The barrier can be formed via techniques commonly used in plastics microfabrication including but not limited to injection molding, chemical deposition photolithography, silkscreening and chemical and/or mechanical machining. Upon cleavage 602 of a construct 601, a portion of the construct 619 remains on the first surface and the cleavage product comprising the affinity region 605 and the detectable marker 609 diffuses and binds to the corresponding capture agent 617 in direct proximity on the second surface 615.

Assay Tools: Single Surface Configuration

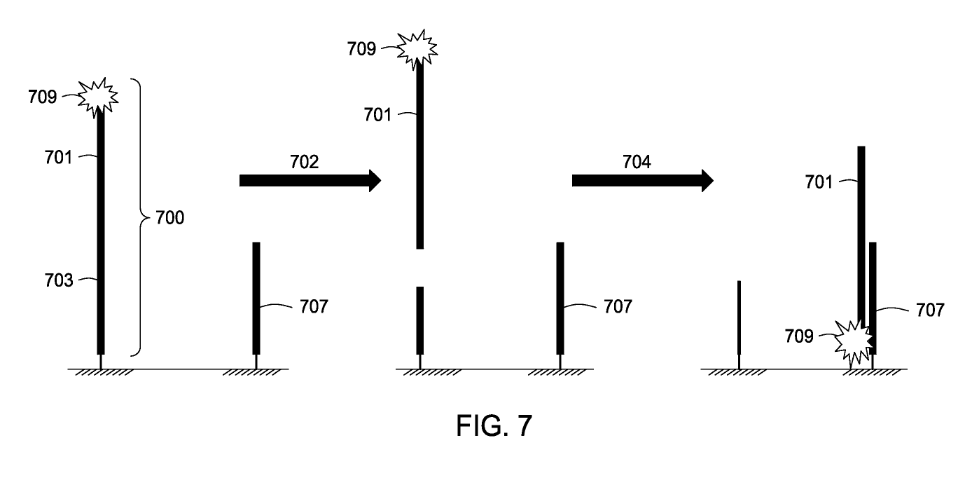

In other aspects of the invention, both the construct and the capture agents are immobilized on discrete regions of a single surface. Different specific examples of such aspects of the cleavage-based assay tools are illustrated in FIGS. 7-12. Although these various aspects are illustrated as one functionalized surface, the assay tool itself will preferably have a second, inert surface that will serve to create a partially or fully enclosed chamber for the capture of the reagents and sample being tested. Thus, the term “single surface configuration” is meant to include the use of other surfaces, such as a glass slide or a cover slip, which can serve a practical function in performing the assay (e.g., conservation of reagents or of detection using optical means, sample loading, wettability), but that do not comprise a construct or a capture agent of the invention.

FIG. 7 is a schematic illustrating the general components of a single surface assay tool of the invention using a single detectable marker. The surface comprises a set of constructs 700 with an enzyme substrate 703, a detectable marker 709 and an affinity region 701 for binding to a capture agent 707. The capture agents with affinity for specific constructs 700 are immobilized in proximity to their corresponding constructs, and preferably in a discrete region adjacent to its corresponding construct. Upon cleavage of the enzyme substrate 703 of the construct 700, the affinity region 701 and the detectable marker 709 are released 702 from the surface. The released affinity region and detectable marker diffuse 704 to the corresponding, proximal capture agent 707, and bind to this moiety to generate a positive signal at the site of the capture agent 707. In certain aspects, the region that binds to the affinity region of the constructs 700 may overlap all or a portion of the enzyme substrate. In addition, the constructs may optionally be attached to the surface with a linker molecule, and in specific aspects a cleavable linker.

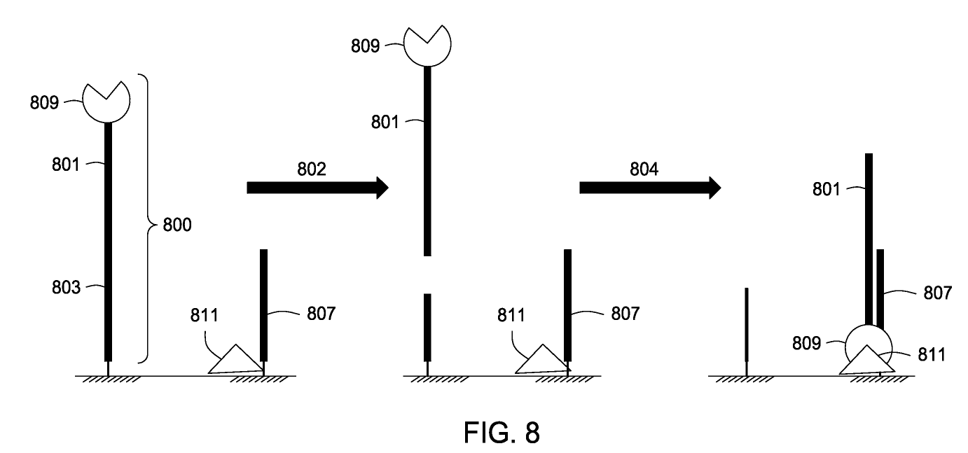

FIG. 8 is a schematic illustrating the general components of a single surface assay tool of the invention using a two molecule detection scheme. As in FIG. 7, the surface comprises a set of constructs 800 with an enzyme substrate 803, a first detectable marker 809 and an affinity region 801 for binding to a capture agent 807. The detector moieties with affinity for specific constructs are immobilized in proximity to their corresponding constructs, and preferably in a discrete region adjacent to its corresponding construct. The detector moieties 807 also comprise a second detectable marker 811 that interact with the first detectable marker 809 to produce a signal, e.g., a FRET-based signal. Upon cleavage of the enzyme substrate 803 of the construct, the affinity region 801 and the first detectable marker 809 are released 802 from the surface. The released affinity region 801 and first component of a detectable marker 809 diffuse 804 to the corresponding, proximal capture agent 807, and bind to this moiety to generate a positive signal at the site of the capture agent 807 through interaction of the first and second components of the combined detectable marker.

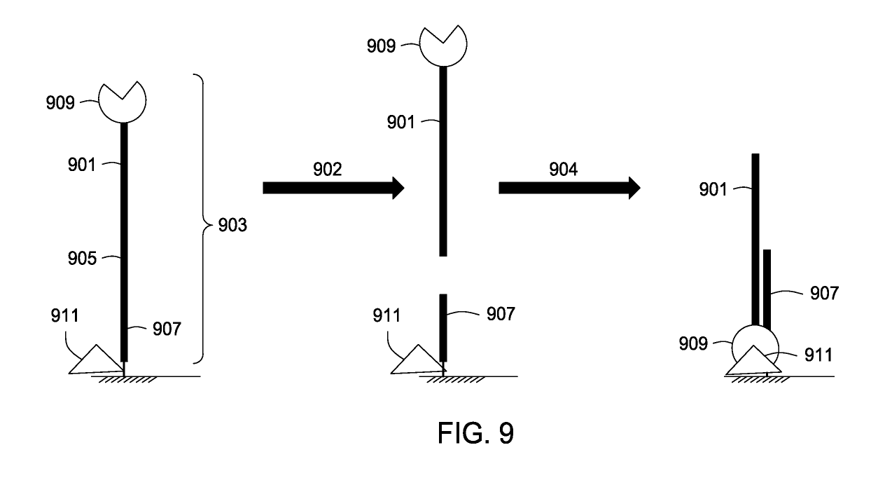

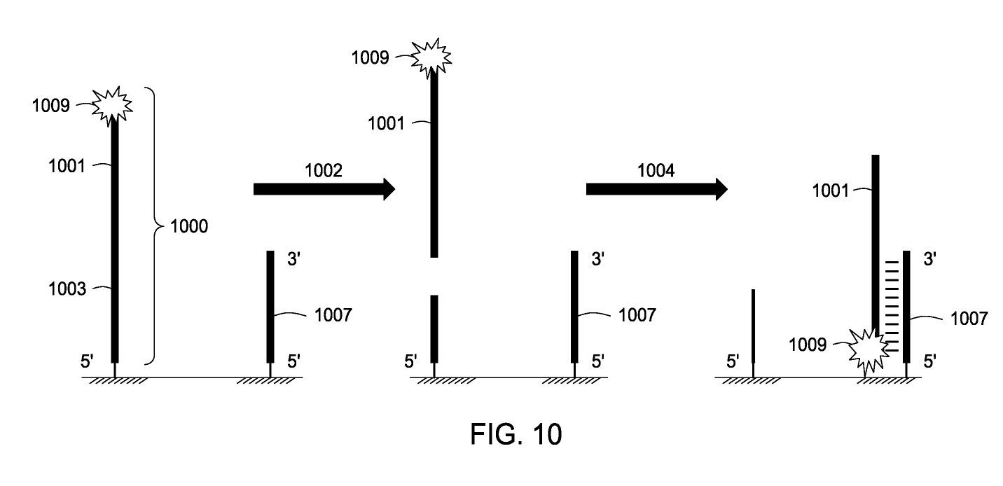

FIG. 9 is a schematic illustrating the general components of another single surface assay tool of the invention using a two molecule detection scheme and a combined construct and capture agent. The surface comprises a set of constructs 903 with an enzyme substrate 905, a first component of a detectable marker 909 and an affinity region 901 for binding to the capture agent region 907, which is located toward the surface on the construct. The construct 903 also comprises a second component of the combined detectable marker 911 associated with the capture agent portion 907 of the construct. When the construct is cleaved 902, the top portion of the construct comprising the affinity region 901 and the first component of the detectable marker 909 are released, and this free component can then bind 904 to the remaining immobilized capture agent, allowing the first and second components of the detectable marker 909 to generate a signal, e.g., a FRET-based signal. In such an aspect, the construct 909 and/or the configuration of constructs and capture agents must be designed so that the construct will not interact with a non-corresponding detectable marker, or interact with a corresponding detectable marker in the absence of a cleavage agent. The construct and/or the configuration of the surface elements must therefore ensure this, e.g., the construct must be designed to be inflexible, the spacing between the constructs and the detectable markers must be sufficient to prevent such inadvertent interaction, and the like. In one specific aspect of the invention, the affinity region and the capture agent comprise complementary nucleic acids that hybridize upon cleavage of a peptide substrate. FIG. 10 illustrates this aspect of the invention. The surface comprises a set of constructs 1000 with a protease substrate 1003, a detectable marker 1009 and a nucleic acid affinity region 1001 for binding to an oligonucleotide capture agent 1007. The oligonucleotide is detector moieties 1007 are immobilized in proximity to their corresponding constructs with complementary nucleic acid affinity regions, and preferably the oligonucleotides are immobilized in a discrete region adjacent to their corresponding constructs. Upon cleavage of the enzyme substrate 1003 of the construct, the affinity region 1001 and the detectable marker 1009 are released 1002 from the surface. The released nucleic acid affinity region 1001 and detectable marker 1009 diffuse 1004 to the corresponding, proximal capture agent 1007, and bind to this moiety to generate a positive signal at the site of the capture agent 1007.

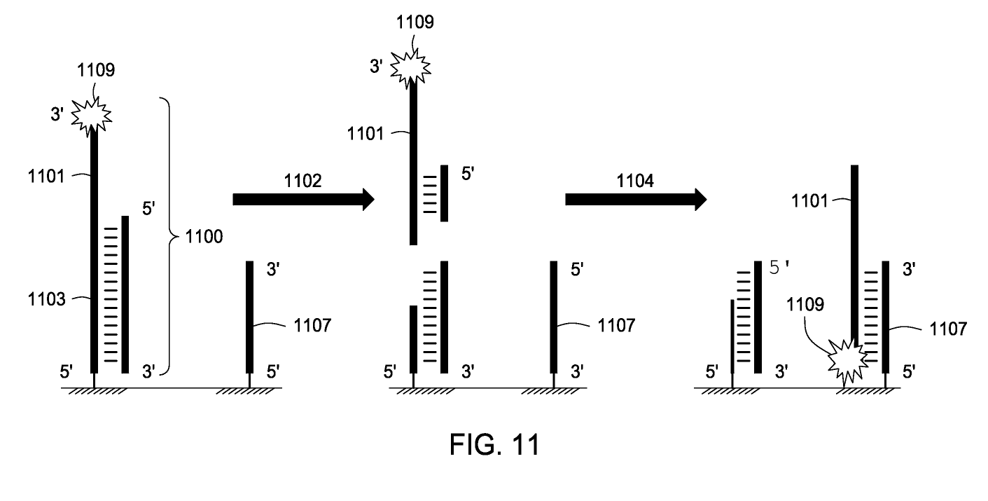

In another specific aspect of the invention, the affinity region and the capture agent comprise complementary nucleic acids that hybridize upon enzymatic cleavage of a nucleic acid substrate, e.g., by cleavage with a restriction endonuclease. FIG. 11 illustrates this aspect of the invention. The surface comprises a set of constructs 1100 with a nucleic acid enzyme substrate 1103, a capture agent 1109 and a nucleic acid affinity region 1101 for binding to an oligonucleotide capture agent 1107. The oligonucleotide capture agents 1107 are immobilized in proximity to their corresponding constructs with complementary nucleic acid affinity regions. Upon cleavage of the enzyme substrate 1103 of the construct 1100, the affinity region 1101 and the detectable marker 1109 are released 1102 from the surface. The released nucleic acid affinity region 1101 and detectable marker 1109 diffuse 1104 to the corresponding, proximal capture agent 1107, and bind to this moiety to generate a positive signal at the site of the capture agent 1107.

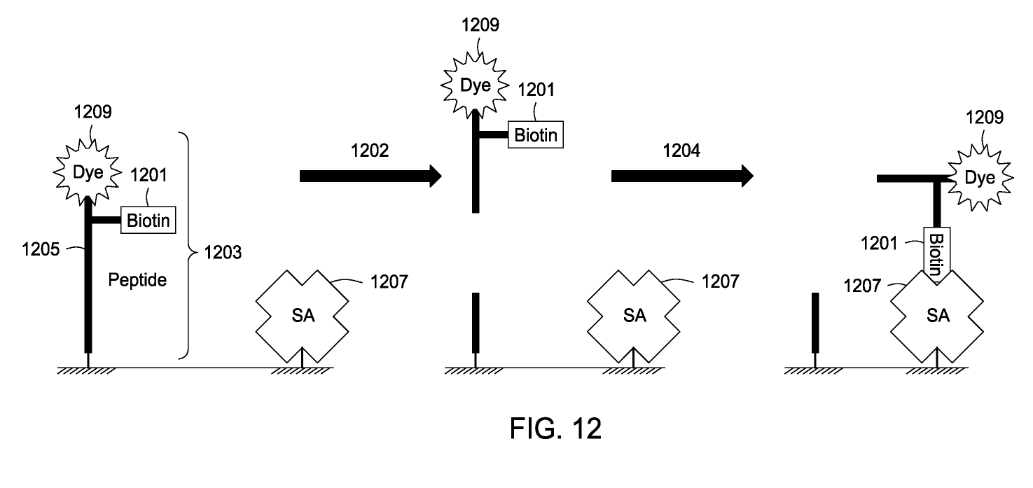

In yet another specific aspect of the invention, the affinity region and the capture agent comprise members of a protein binding pair that specifically bind to one another with high affinity upon enzymatic cleavage of a peptide substrate, e.g., by cleavage with a protease. FIG. 12 illustrates this aspect of the invention. The surface comprises a set of constructs 1203 with a peptide substrate 1205, a capture agent 1209 and a protein binding pair member (here, biotin) 1201 for binding to a capture moiety (here streptavidin) 1207. The peptide detector moieties 1207 are immobilized in proximity to their corresponding binding pair constructs, and preferably are immobilized in a discrete region adjacent to their corresponding binding pair constructs. Upon cleavage of the peptide substrate 1205 of the construct 1203, the affinity region 1201 and the detectable marker 1209 are released 1202 from the surface. The released peptide affinity region 1201 and detectable marker 1209 diffuse 1204 to the corresponding, proximal peptide capture agent 1207, and bind to this moiety to generate a positive signal at the site of the capture agent 1207.

Competitive Binding Assays

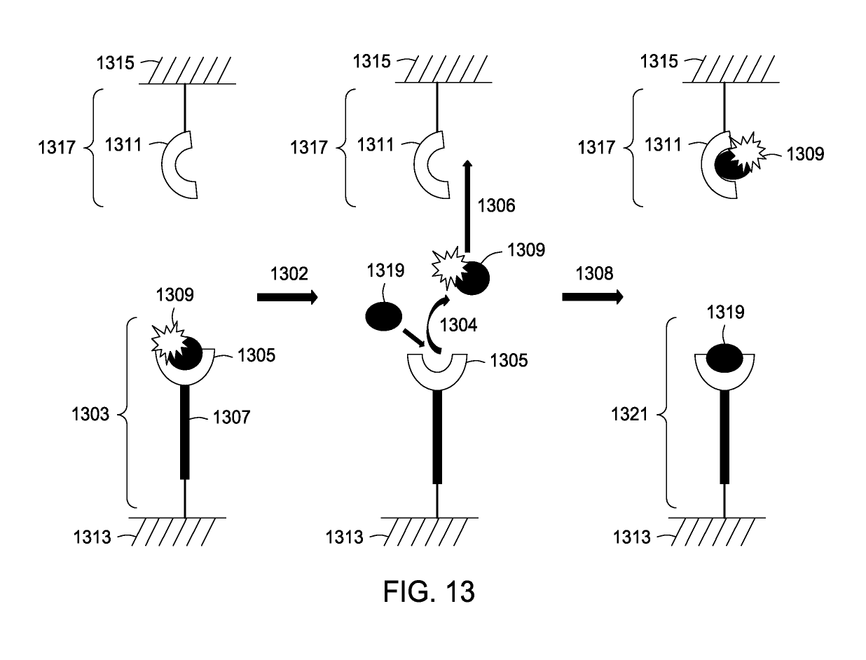

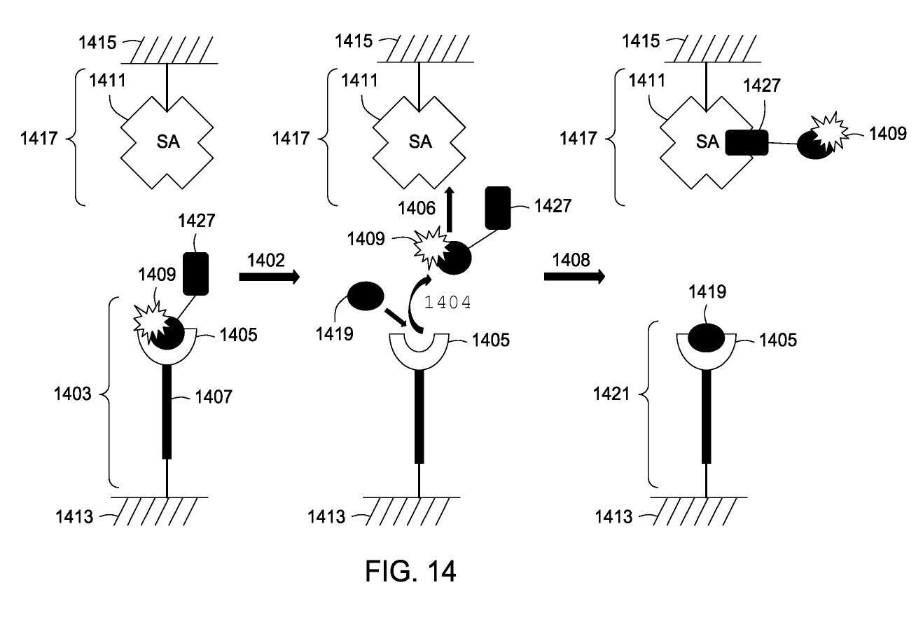

In certain aspects, the assay tool of the invention can be used to detect competitive binding of a region of interest. The binding molecule is shown in the following figures as a single component, but is meant to encompass binding molecules with various components. For example, to facilitate the analysis of a variety of different molecules with a single capture agent, the bound molecule that is displaced by the agent of interest in a sample could be chimeric, having a portion that is specific for binding to a capture agent. This binding portion could be a peptide, a small molecule, or any other member of a binding pair (e.g., biotin). Examples of this aspect are illustrated in FIGS. 13-15.

In FIG. 13, the affinity region 1305 of a construct 1303 immobilized to a first surface 1313 comprises a bound first binding molecule 1309 at the binding area of interest on the construct. The capture agent 1317 comprises a capture molecule 1311 immobilized to a second surface 1315. Upon introduction 1302 of a second binding molecule 1319 that has a higher affinity to the affinity region 1305 than the first binding molecule 1309, the bound molecule 1309 is released 1308 and the new binding molecule binds in its place in the affinity region 1305. The released molecule 1309 diffuses 1306 to the corresponding capture agent 1311, and binds to this moiety 1311. The first binding molecule 1309 is itself labeled by a detectable marker, and this can be directly detected on the second surface upon binding of the molecule 1309 to the capture agent 1311.

In another aspect, the first binding molecule is directly labeled, and both are associated with a third component that will specifically bind to the capture agent. The affinity region 1405 of a construct 1403 immobilized to a first surface 1413 comprises a bound first binding molecule 1409 at the binding area of interest on the construct that is associated with a component of a binding pair 1427 that specifically binds to the capture molecule 1411 of the capture agent 1417. Here the capture molecule is streptavidin, and the molecule associated with the first binding molecule is biotin, although any member of a reactive pair can be used. The capture agent 1417 comprises a capture molecule 1411 immobilized to a second surface 1415. Upon introduction 1402 of a second binding molecule 1419 that has a higher affinity to the affinity region 1405 than the first binding molecule 1409, the bound molecule 1409 is released 1408 and the new binding molecule binds in its place in the affinity region 1405. The released molecule 1409 and the biotin 1427 diffuse 1406 to the corresponding capture agent 1411, and binds to the streptavidin 1411. The first binding molecule 1409 is itself labeled by a detectable marker, and this can be directly detected on the second surface upon binding of the biotin 1427 to the streptavidin 1411.

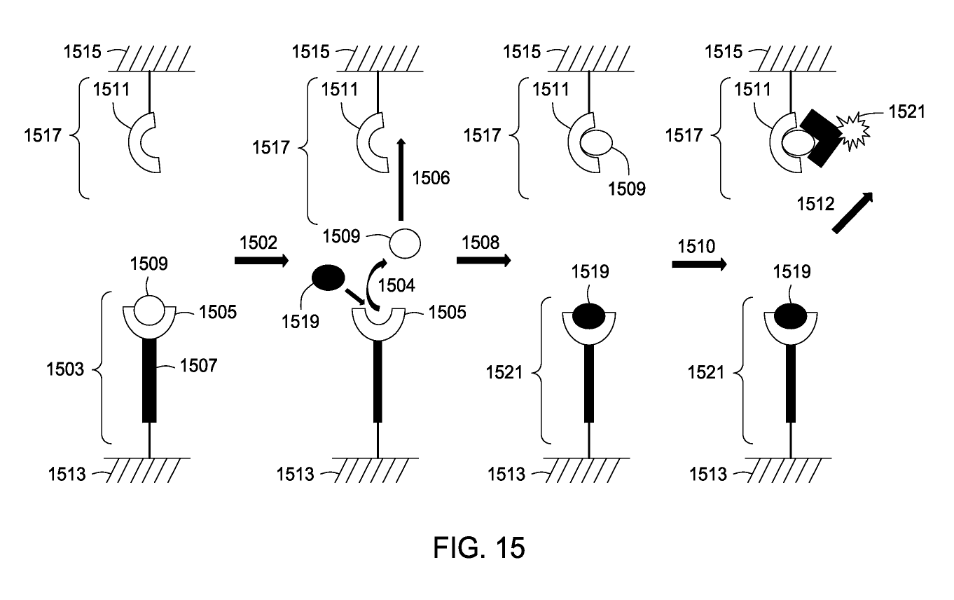

In yet another aspect illustrated in FIG. 15, the affinity region 1505 of a construct 1503 immobilized to a first surface 1513 comprises a bound first binding molecule 1509 at the binding area of interest on the construct. The capture agent 1517 comprises a capture molecule 1511 immobilized to a second surface 1515. Upon introduction 1502 is of a second binding molecule 1519 that has a higher affinity to the affinity region 1505 than the first binding molecule 1509, the bound molecule 1509 is released 1508 and the new binding molecule binds in its place in the affinity region 1505. The released molecule 1509 diffuses 1506 to the corresponding capture agent 1511, and binds to this moiety 1511. The first binding molecule is not directly labeled in this aspect, but rather is detected by the introduction 1512 of a detection molecule 1521 that specifically binds to the released molecule 1509, which binds to the molecule:capture agent complex and generates a positive signal at the site of the capture agent 1517.

In certain aspects of the invention, both double surface aspects and single surface aspects, it may be desirable to use a surface with, for example, wells, raised regions, pedestals, etched holes, or the like. These may be designed to optimize the process of diffusion, to prevent the likelihood of cross-contamination, to optimize distances for purposes of detection of the cleavage activity, and the like.

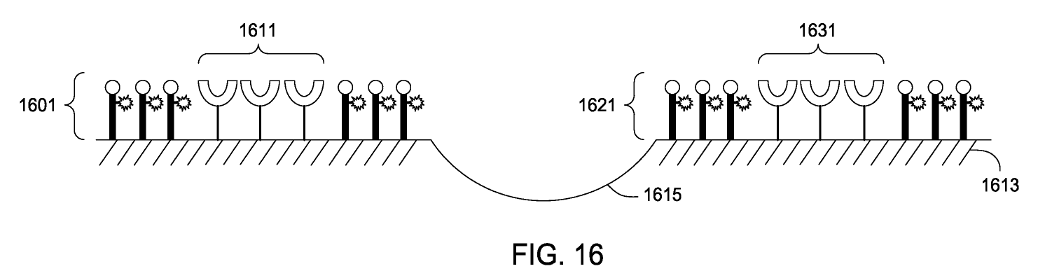

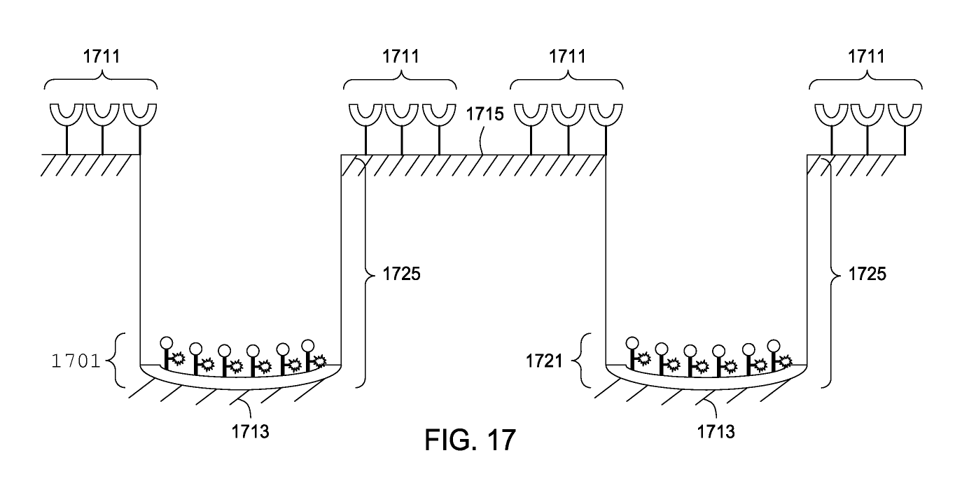

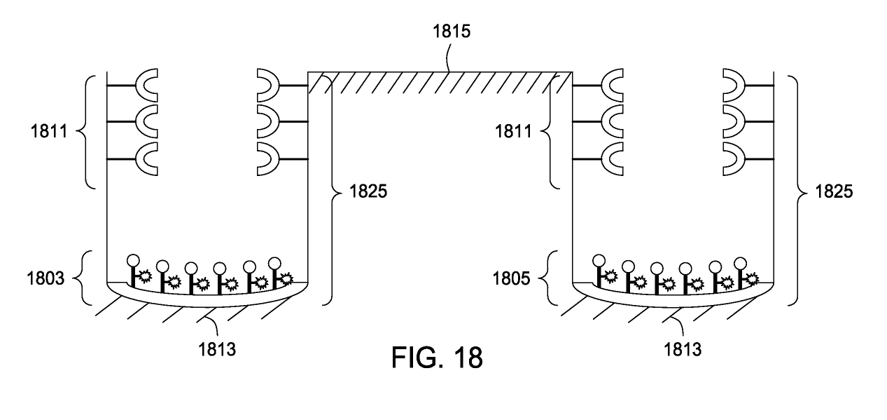

In certain aspects of the invention, one of the surfaces of the tool comprises such surface aspects. In one specific example, illustrated in FIG. 16, the assay tool is constructed of a first patterned surface 1613 having a set of two or more immobilized constructs 1601, 1621, each with corresponding capture agents 1611, 1631 In certain other aspects (not shown), it may be preferable to have just one capture agent at both locations, i.e. 1611 and one or more regions of depressed surface 1615 between the constructs 1601, 1621. In another example, illustrated in FIG. 17, the assay tool comprises a first surface 1715 with a series of wells 1725 patterned thereon. The bottom of the wells 1713 comprise a set of two or more immobilized constructs 1701, 1721, and either a set of single capture agents 1711 corresponding to a common affinity agent or different corresponding capture agents for particular constructs (not shown) located on the surface above each well. In yet another example, illustrated in FIG. 18, the assay tool comprises a first surface 1815 with a series of wells 1825 patterned thereon. The bottom of the wells 1813 comprise a set of two or more immobilized constructs 1803, 1805 that can be patterned using a multitude of methods including but not limited to non-contact dispensing (piezzo, solenoid), molecular printing or predeposition prior to imobilization of either a set of single capture agents 1811 corresponding to a common affinity agent or different, corresponding capture agents for particular constructs (not shown) located physically above the constructs in the wells.

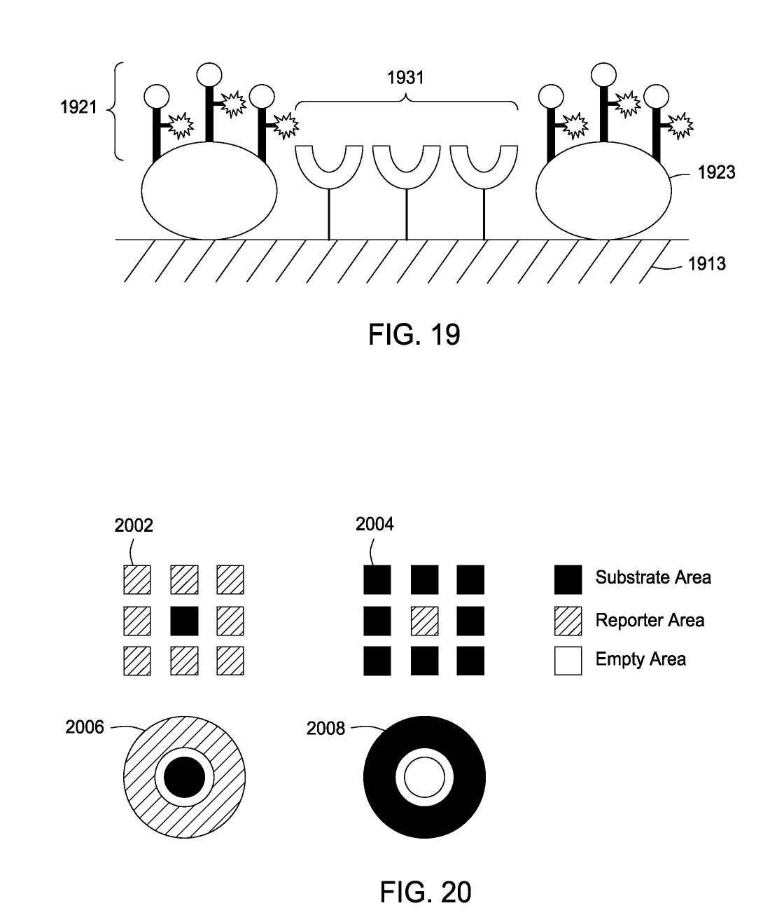

In some aspects, one of the surfaces of the assay tool may comprise beads to which the constructs and/or capture agents are immobilized. These beads may be associated with a patterned surface, e.g., placed in wells, or they may be associated with a planar surface so that the beads effectively are raised surfaces on the planar surface. In one example of this aspect, illustrated in FIG. 19, a series of beads 1923 having immobilized constructs 1921 are located on a planar surface 1913 in proximity to corresponding capture agents 1931. Cleavage and detection of such cleavage is essentially as set out in the previous figures.

In one preferred aspect, constructs are immobilized onto beads, which are located in a well located on a planar surface. In a specific aspect, the well comprises capture agents immobilized to the surface of the well in which the bead sits. Following cleavage, the detectable agents from the constructs on the bead are captured on the surface of the well. The beads are then optionally removed and the positive signal on the surface of the wells is detected.

In another aspect, the constructs are immobilized on the well surface and the capture agents are immobilized on beads located in the well. Following cleavage, the detectable markers are released from the well surface and bind to the capture agents on the beads. The positive signal created from the binding of the detectable label to the capture agent on the beads can be detected, e.g., using a technique such as flow cytometry. In a specific example, positive signal on the beads in each well can be read serially (and thus identified by well location) and/or the beads can be coded (e.g., using xMAP™ technology (Luminex Corp. Austin, Tex.) or VeraCode™ technology (Illumina, San Diego, Calif.)) to enable detection of multiple activities in a single assay, which can be carried out separately in wells, and optionally pooled following detection to determine activity.

In yet another aspect, the wells can be a passive surface and two or more beads optionally labeled as above with different immobilized constructs and/or capture agents can be mixed in individual wells. For example, an individual well may comprise a bead having an immobilized construct and a bead having an immobilized capture agent that upon cleavage of a substrate or competitive binding of a substrate on one bead is transferred to the capture agent of the other bead and subsequently detected by generation of a signal that can be associated with the bead label.

In certain aspects of the invention, fluid flow or other types of active transport, e.g., electrical force is used to assist in the diffusion of a cleavage product or a molecule that has been otherwise removed from a construct, e.g., through competitive binding. In such aspects, the capture agents corresponding to the different constructs will generally be located downstream of the constructs based on fluid flow. These aspects may be combined with the aspects comprising the spacing units to avoid potential cross-contamination between different constructs and capture agents.

In aspects of the invention using a single surface, it may also be useful to have multiple copies of a single enzyme substrate and/or multiple copies of the capture agent on the surface, e.g., to increase signal to noise through averaging. For example, multiple features comprising multiple individual copies of a construct may be used. In other examples, multiple copies of the same molecule can be used on the surface in different regions. In some specific aspects, illustrated in FIG. 20, one region is surrounded by adjacent regions to maximize the detection area (or “reporter region”). The aspects include: discrete reporter regions surrounding a single substrate region 2002, discrete substrate regions surrounding a single reporter region 2004, a single reporter region surrounded by a continuous region of substrate 2006, and a single substrate region surrounded by a continuous region of reporter 2008. The empty regions between the substrate region and the reporter region are optional, and may depend on the limits of the particular detection mechanism used to detect the capture agent on the surface.

In aspects which include a depressed region on the surface, e.g., a well, the substrate area can be located within the well (and preferably at the bottom of the well) and the reporter area could be the sides of the well, either at the top surrounding the well or actually within the well itself to maximize capture of the cleavage product. In this configuration, the bottom illustrations of FIG. 20 may represent the sides and/or bottom of a well.

Other aspects will become apparent to one skilled in the art upon reading the present disclosure—the one common attribute is that the reporter area can be directly associated with cleavage of a substrate upon exposure to enzymatic activity in a sample.

Using Mass-Spectrometry (MS) to Identify Cleavage Sites

In certain circumstances, it is important to identify the positions of the protease cleavage on a peptide substrate following confirmation of protease activity. In certain aspects of the invention, the position of the cleavage site can be detected using mass-spectrometer (“MS”) detection on the region of the array with the enzyme substrate. After an assay is done, MS can be utilized to image the reporter areas to obtain the assay data followed by MS measurements. Such imaging can be used to provide information regarding the molecular weight of the cleaved fragments left following, e.g., protease cleavage of an enzyme substrate. Therefore the cleavage place within the peptide substrate can be identified. There are several publications describing the use of microarrays in combination with MS technology (Isola et al, 2001) (Brandt et al, 2003) (Yu et al, 2006) that demonstrate that the molecular weight of DNA fragments hybridized to complementary sequences on the microarray surface can be determined using mass spectrometry.

Enzymes for use with the Present Invention

The present tools and methods are useful to detect the activity of any enzyme that directly or indirectly leads to a cleavage event. Enzymes that can be tested using the tools and methods of the invention include, but are not limited to, proteases, phosphatases, nickases, nucleases, and glycosylases.

Proteases

Serine proteases are inhibited by a diverse group of inhibitors, including synthetic chemical inhibitors for research or therapeutic purposes, and also natural proteinaceous inhibitors. One family of natural inhibitors called “serpins” (abbreviated from serine protease inhibitors) can form a covalent bond with the serine protease, inhibiting its function. The best-studied serpins are antithrombin and alpha 1-antitrypsin, studied for their role in coagulation/thrombosis and emphysema/A1AT respectively. Artificial irreversible small molecule inhibitors include AEBSF and PMSF.

The proteasome hydrolases constitute a unique family of threonine proteases. A conserved N-terminal threonine is involved in catalysis at each active site. The three catalytic subunits are synthesized as pre-proteins. They are activated when the N-terminus is cleaved off, making threonine the N-terminal residue. Catalytic threonines are exposed at the lumenal surface.

Cysteine proteases have a common catalytic mechanism that involves a nucleophilic cysteine thiol in a catalytic triad. The first step is deprotonation of a thiol in the enzyme's active site by an adjacent amino acid with a basic side chain, usually a histidine residue. The next step is nucleophilic attack by the deprotonated cysteine's anionic sulfur on the substrate carbonyl carbon. In this step, a fragment of the substrate is released with an amine terminus, the histidine residue in the protease is restored to its deprotonated form, and a thioester intermediate linking the new carboxy-terminus of the substrate to the cysteine thiol is formed. The thioester bond is subsequently hydrolyzed to generate a carboxylic acid moiety on the remaining substrate fragment, while regenerating the free enzyme. Examples of cysteine proteases include papain, cathepsins, caspases, and calpains.

Aspartic proteases are a family of eukaryotic protease enzymes that utilize an aspartate residue for catalysis of their peptide substrates. In general, they have two highly-conserved aspartates in the active site and are optimally active at acidic pH. Nearly all known aspartyl proteases are inhibited by pepstatin.

Eukaryotic aspartic proteases include pepsins, cathepsins, and renins. They have a two-domain structure, probably arising from ancestral duplication. Retroviral and retrotransposon proteases are much smaller and appear to be homologous to a single domain of the eukaryotic aspartyl proteases.

Metalloproteinases are a family of protein-hydrolyzing endopeptidases that contain zinc ions as part of the active structure. There are two subgroups of metalloproteinases: metalloexopeptidases and metalloendopeptidases. Well known metalloendopeptidases include ADAM proteins and matrix metalloproteinases.

Phosphatases

A phosphatase is an enzyme that removes a phosphate group from its substrate by hydrolysing phosphoric acid monoesters into a phosphate ion and a molecule with a free hydroxyl group. In certain circumstances, a phosphatase can modify a peptide substrates susceptibility to another enzyme, e.g., a protease, by removing a protective phosphate. For example, removal of the phosphate on the molecule Alpha II spectrin makes it susceptible to cleavage by calpain, a ubiquitous Ca²⁺-dependent proteases. Nicolas G et al., Molecular and Cellular Biology, May 2002, p. 3527-3536, Vol. 22, No. 10.

Phosphatases that can be used to potentiate cleavage of the enzyme substrates for use in the constructs of the present invention can be categorized by their substrate specificity. They include, but are not limited to, tyrosine-specific phosphatases, serine/threonine specific phosphatases, dual specificity phosphatases (which recognize phospho-tyrosine/serine/threonine, histidine phosphatase and lipid phosphatase.

DNA Glycosylases

In some aspects, DNA glycosylases can be used to remove a wide range of polynucleotide bases from an enzyme substrate by cleaving the N-glycosylic bond between the base and deoxyribose, leaving an abasic site (see, e.g., Krokan et. al. (1997) Biochem. J. 325:1-16). DNA glycosylases for use with the invention include, but are not limited to, uracil-DNA glycosylases, G/T(U) mismatch DNA glycosylases, alkylbase-DNA glycosylases, 5-methylcytosine DNA glycosylases, adenine-specific mismatch-DNA glycosylases, oxidized pyrimidine-specific DNA glycosylases, oxidized purine-specific DNA glycosylases, EndoVIII, EndoIX, hydroxymethyl DNA glycosylases, formyluracil-DNA glycosylases, pyrimidine-dimer DNA glycosylases.

In certain aspects, a uracil may be synthetically incorporated in a nucleic acid substrate to replace a thymine, and the uracil can be removed by treatment with uracil DNA glycosylase (see, e.g., Kunkel, T. A. (1985) Proc. Natl. Acad. Sci. USA 82:488-492; Lindahl (1990) Mutat. Res. 238:305-311; Published U.S. Patent Application No. 20050208538).

For each of the above aspects, the abasic site on the nucleic acid strand may then be cleaved by E. coli Endonuclease IV.

Restriction Endonucleases

In certain aspects of the invention, the constructs used in the tools and methods of the invention comprise cleavage sites for restriction endonucleases. A restriction endonuclease is an enzyme that can cut at a double-stranded or single stranded nucleic acid enzyme substrate at a specific recognition nucleotide sequences known as restriction sites. The tools and methods of the invention can detect restriction endonuclease activity within a sample utilizing constructs that comprise nucleic acid enzyme substrates having specific restriction sites for restriction endonuclease cleavage. Restriction endonucleases are categorized into three general groups (Types I, II and III) based on their composition and enzyme cofactor requirements, the nature of their target sequence, and the position of their DNA cleavage site relative to the target sequence. Examples of restriction endonucleases for use in cleavage of the enzyme substrates of the invention include, but are not limited to, those disclosed in REBASE, the restriction endonuclease database at http://rebase.neb.com/rebase/rebase.html.

In some aspects, the enzyme substrate of the invention comprises a site for cleavage by a Type I endonuclease. These enzymes cut at a site that differs, and is some distance (at least 1000 bp) away, from their recognition site. The recognition site is asymmetrical and is composed of two portions—one containing 3-4 nucleotides, and another containing 4-5 nucleotides—separated by a spacer of about 6-8 nucleotides.

In other aspects, the enzyme substrate of the invention comprises a site for cleavage by a Type II endonuclease. More than 3000 type II restriction endonucleases have been discovered. They recognize short, usually palindromic, sequences of 4-8 by and, in the presence of Mg²⁺, cleave the DNA within or in close proximity to the recognition sequence. The orthodox type II enzymes are homodimers which recognize palindromic sites.

In certain other aspects, the enzyme substrate of the invention comprises a site for cleavage by a Type IIS endonuclease. Exemplary Type IIs restriction endonucleases include, but are not limited to, Eco57M I, Mme I, Acu I, Bpm I, BceA I, Bbv I, BciV I, BpuE I, BseM II, BseR I, Bsg I, BsmF I, BtgZ I, Eci I, EcoP15 I, Eco57M I, Fok I, Hga I, Hph I, Mbo II, Mnl I, SfaN I, TspDT I, TspDW I, Taq II, and the like.

Nickases

In related aspects, a nickase can be used to cleave a double stranded nucleic acid enzyme substrate. Nickases are endonucleases that recognize a specific recognition sequence in double stranded DNA, and cut one strand at a specific location relative to said recognition sequence, thereby giving rise to single-stranded breaks in double-stranded nucleic acids. Once the nickase has cleaved a strand of the substrate, the assay conditions may be altered to provide denaturation of the double stranded substrate, and the nicked strand will be free to diffuse to the capture agent while the remainder of the nucleic acid molecule will remain immobilized to the surface. Nickases include but are not limited to Nb.BsrDI, Nb.BsmI, Nt.BbvCI, Nb.BbvCI, Nb.BtsI and Nt.BstNBI.

Binding Pair Affinities

Peptide Binding Pairs

The strength of the interaction of a peptide binding pair can be characterized by its “binding affinity” of one part of the binding pair to a given binding site or epitope on the other member of the binding pair. For example, in the field of immunology, antibodies are characterized by their “binding affinity” to a given binding site or epitope. Every antibody is comprised of a particular 3-dimensional structure of amino acids, which binds to another structure referred to as an epitope or antigen.

The selective binding of a binding partner to a composition is a simple bimolecular, reversible reaction, not unlike the binding of an antibody to its antigen. For example, if the antibody is represented by Ab and the antigen by Ag, the reaction can be analyzed by standard kinetic theory. Assuming a single binding site the reaction is represented by the equation I as follows:

$$Ag + Ab {⇌}_{{k}_{2}}^{{k}_{1}} Ag - Ab \qquad I .$$

where Ag-Ab is the bound complex. The forward and reverse binding reactions are represented by rate constants k₁ and k₂ respectively. The “binding affinity” of the antibody to the antigen is measured by the ratio of complexed to free reactants at equilibrium. The lower the concentration of the reactants at equilibrium, the higher the binding affinity of the antibody for the antigen. In the field of immunology, the binding affinity is represented by an “affinity constant” which is represented by the symbol “K” or sometimes referred to as “Kₐ”. The “K” is defined by the equation II as follows:

$$K = \frac{[ Ag - Ab ]}{[ Ag ] [ Ab ]} = \frac{{k}_{1}}{{k}_{2}} \qquad II .$$

where the brackets denote concentration in moles per liter or liters per mole.

A typical value for the binding affinity Kₐ which is also referred to as “K” and is the “affinity constant” which for a typical antibody is in a range of from about 10⁵ to is about 10¹¹ liters per mole. The Kₐ is the concentration of free antigen needed to fill half the binding sites of the antibody present in solution with the antigen. If measured in liters per mole a higher Kₐ (e.g. 10¹¹) or higher affinity constant indicates a large volume of solvent, a very dilute concentration of free antigen, and as such indicates the antibody has a high binding affinity for the epitope.

If the Kₐ is measured in moles per liter a low Kₐ (e.g. 10⁻¹¹) indicates a less concentrated solution of the free antigen needed to occupy half of the antibody binding sites, and as such a high binding affinity.

Equilibrium is achieved in order to measure the Kₐ. More specifically, the Kₐ is measured when the concentration of antibody bound to antigen [Ag-Ab] is equal to the concentration of the antibody [Ab]. Thus, [Ag-Ab] divided by [Ab] is equal to one. Knowing this, the equation II above can be resolved to the equation III as follows:

$$K = \frac{1}{[ Ag ]} \qquad III .$$

In equation III the units for K are liters per mole. Typical values in liters per mole are in a range of from about 10⁵ to about 10¹¹ liters per mole.

The inverse of the above equation is K=[Ag] where the units for K are in moles per liter, and the typical values are in a range of 10⁻⁶ to 10⁻¹² moles per liter.

The above shows that typical binding affinities can vary over six orders of magnitude. Thus, what might be considered a useful antibody might have 100,000 times greater binding affinity as compared to the binding affinity of what might be considered a different antibody, which is also considered useful.

Nucleic Acid Binding Pairs

The degree of complementarity between nucleic acid binding pairs and the base composition of those pairs will determine the Tₘ of the duplex, and thus the strength of the hybridization between the members of the pair. Hybridization refers to the process in which two single-stranded polynucleotides bind non-covalently to form a stable double-stranded polynucleotide. The nucleic acid binding pairs for use in the present invention preferably hybridize under stringent conditions, i.e., conditions under which a binding partner will selectively to its binding pair mate. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5.times. SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning: A laboratory Manual” 2.sup.nd Ed. Cold Spring Harbor Press (1989) and Anderson “Nucleic Acid Hybridization” 1.sup.st Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that is sequence is present in a complex mixture (e.g., a sample comprising cellular DNA and/or RNA).

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

### Peptide Synthesis

Peptides were synthesized by manual Fmoc-based peptide synthesis in fritted syringes (Fields et al., Biochemistry. 1990 Jul. 17; 29(28):6670-7; Chan D et al., J Cell Physiol. 2000 December; 185(3):339-47) on Fmoc-glycine Wang resin solid support (Advanced Chemtech). Fmoc-Lpropargylglycine (Advanced Chemtech) was used at the first step of the peptide synthesis to introduce the alkyne group at the C-terminus of peptides. Fmoc-Lys(biotin)-OH (EMD Chemicals) was used as the last amino acid to introduce the N-terminal biotin residue. Peptide synthesis was followed by Fmoc deprotection and coupling of the fluorescent dye (6-carboxy-tetramethylrhodamine, TAMRA, EMD Chemicals). Molecular weight of peptides were confirmed using a ProteinPlex SELDI laser desorption/ionization TOF mass spectrometry based analytical system (Bio-Rad). We used peptide substrates GAENLYFQGA and GALVPRGSAG targeting TEV and thrombin proteases as model peptides. The protease recognition sites are shown in bold. N- and C-terminal amino acid residues were added to the ends of these peptides to create additional space for more efficient protease binding.

Peptides were attached to a solid support via their C-terminus as this orientation is known to work well in protease assays (Salisbury C M et al., J Am Chem. Soc. 2002 Dec. 18; 124(50):14868-70.2002). The obtained peptide substrates were confirmed to cleave specifically by TEV and thrombin proteases in solution. Individual peptides were cleaved in solution and the products of the cleavage reactions were analyzed using laser desorption/ionization TOF mass spectrometry. The molecular weights were in agreement with calculated values. Complete cleavage of the TEV peptide substrate by TEV protease (20 U/100 ul) was confirmed while the thrombin peptide substrate remained intact. Thrombin protease (200 nM) specifically cleaved the thrombin peptide whereas there was no detectable cleavage of the TEV peptide.

### Example 2

### Preparation of Substrate Slides

Standard 75×25 mm microscope slides were used to implement the two-surface assay tool. One slide comprises the peptide substrates (the “substrate” slide), and the other slide comprises the capture agents (the “reporter” slide). Together these comprise a two surface assay tool.

Microscope slides with a non-protein polyethyleneglycol (PEG) linker were is obtained in the following way. ES amino slides (Erie Scientific) were treated with 0.1M N3-(PEG)7-COOH(O-(2-Azidoethyl)-O′-(N-diglycolyl-2-aminoethyl) heptaethyleneglycol, EMD Chemicals) solution in DMF containing 0.1M PyBop (EMD Chemicals) and 0.2M N,N-diisopropylethylamine overnight at room temperature. Slides were subsequently washed with DMF (3 times), and water (3 times). This 33-atoms PEG-linker resulted in direct introduction of azide groups on the slide surface. Substrate slides were blocked with a non-protein blocking solution containing Ficoll 400, PVP 40 (polyvinylpyrrolidones), 40 kDa MW), PEG 3350 and 8000 (polyethyleneglycoles), at 0.02% each in 1×PBS for 1 hour at RT to prevent nonspecific peptide absorption during peptide immobilization.

The assay was performed both with the slides in direct contact (no physical spacer was used such that the total volume of the assay determined the spacing between the slides, for example 30 μL will provide a gap of approximately 15 μm.) or with a physical spacer fabricated by die cutting a 90 μm thick polymer sheet. Many methods exist for creating a defined gap between the slides including fabrication of spacers that can be placed between the slides via standard manufacturing process including but not limited to laser cutting, die cutting, machining. Fabrication of a physical spacer directly on the slide can easily be achieved using standard fabrication methods including but not limited to silk-screening, spin coating, photolithography, and micro-fabrication (e.g. removal or addition of material to the surface).

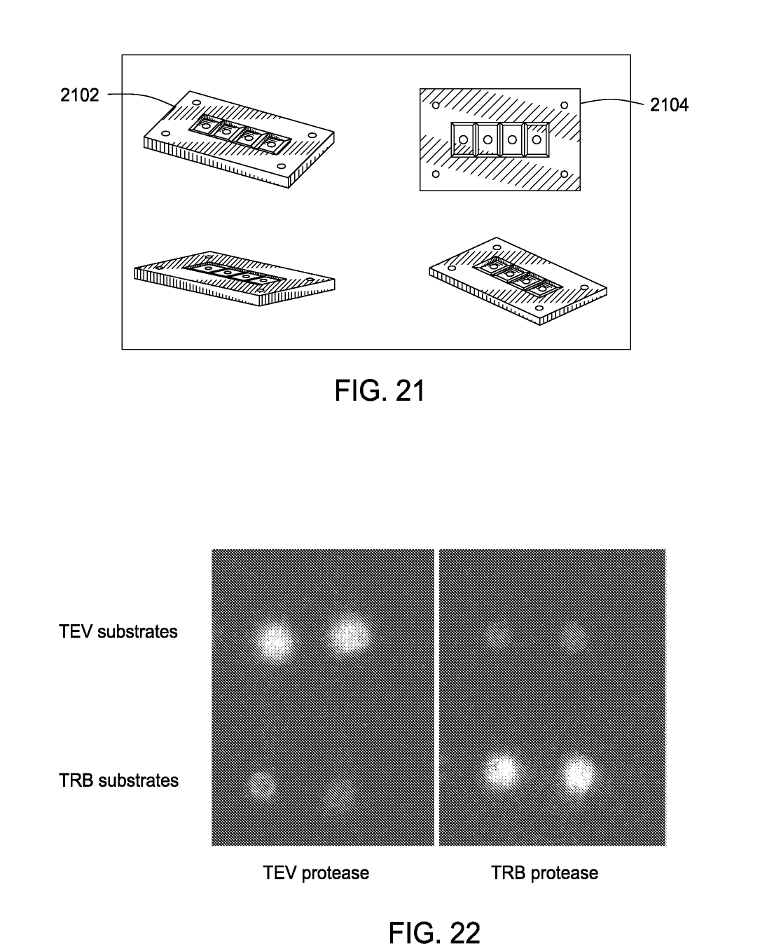

Once the substrate slide preparation and peptide immobilization processes was optimized, the substrate slides were cut into 8 pieces and four of these pieces were used, one per standard size reporter slide, allowing four different protease samples to be run per one reporter slide. The substrate slides were cut with either a regular diamond glass cutter or laser engraving technology for slide cutting, and paper clips were used to hold the two surface assay tool together. The laser had the benefit of allowing a label to be etched on the surface of the substrate slides in order to discriminate the top and bottom sides of the slides. In addition, cartridges were designed and manufactured for the assembly of the assay tool slides (FIG. 21). This allowed quick assembly of the surface pairs, 2102 and 2104, and reduced assay variability and reduce sample volume to 5 μl per substrate slide.

The process of “click chemistry” was used for the immobilization of peptides or DNA-peptide conjugates on microscope slides. Previously obtained published experimental conditions (Kolb H C et al., Angew Chem Int Ed Engl. 2001 Jun. 1; 40(11):2004-2021. H C et al., Drug Discov Today. 2003 Dec. 15; 8(24):1128-37. Review. 2001; Zhang et al., Anal Chem. 2006 Mar. 15; 78(6):2001-8; Loaiza et al., J Comb Chem. 2006 March-April;8(2):252-61; Sohma Y and Kiso Y, Chembiochem. 2006 October; 7(10):1549-57; Rengifo, H. R. et al., Langmuir 2008, 24, 7450-7456.) were further optimized to enable efficient immobilization of alkyne group containing peptides on azide group containing substrate microscope slides. ES amino slides (Erie Scientific) were converted into slides with surface azide groups. We observed that peptide positioning on the solid surface is an important factor in the efficiency of protease cleavage. This is in agreement with previously published data (Macbeath Science, 289:1760 (2000)), as azide slides with a short linker between immobilized peptides and the surface showed very poor cleavage of the peptide substrates by proteases, while the slides with a protein BSA linker or with a PEG linker enabled efficient cleavage of peptides. There was no detectable difference in the cleavage results for BSA and PEG linkers. However, the process for incorporation of the PEG linker is less complicated and less likely to be subject to spurious cleavage. The PEG linker was thus used for the following experiments. Neutravidin coated microscope slides as reporter slides.

### Example 3

### Immobilization of Peptides

The peptides were dissolved at 2 μm concentration in 0.1M Tris-HCl buffer pH 7.5 containing 20% DMSO, 10 mM CuSO4, and 50 mM Na-ascorbate. After spotting, the immobilization reaction was allowed to proceed for 12 hours at room temperature in a humidified chamber. The slides were washed with DMF, DMSO, and 50 mM Tris-HCl, pH 7.5 containing 0.01% Tween 20 for 30 min per washing solution, rinsed with water, ethanol, and dried. The peptides were spotted on the slides (0.1 μl per spot) using manual spotting with a standard 0.1-2 μl laboratory pipettor (Eppendorf). This yielded spots with a diameter of approximately 800 um.

### Example 4

### Preparation of Neutravidin Reporter Slides

Microscope slides containing epoxy groups (Erie Scientific) were treated with neutravidin solution (5 mg/ml, Pierce) in 50 mM Na-carbonate buffer, pH 9.4 for 18 hours at RT in a humidified chamber, blocked with 0.5M ethanolamine solution for 1 hour at RT to inactivate remaining epoxy groups, followed by blocking with solution containing Ficoll 400, PVP 40 (polyvinylpyrrolidones), 40 kDa MW, PEG 3350 and 8000 (polyethylenglycoles), at 0.02% each in 1×PBS for 1 hour at RT to prevent nonspecific protein absorption during our protease assay. The slides were washed three times with water between the immobilization/blocking steps described above. Finally, the slides were washed three times with 1×PBS buffer containing 0.01% Tween-20, three times with 1×PBS, and stored in 1×PBS buffer in the refrigerator until used.

### Example 5

### Detection of Peptide Cleavage Using a GOS Array Pair

A sandwich assay tool was used to show specificity of cleavage and detection using two enzymes, TEV and Thrombin, is shown in FIG. 22: The tool comprises peptide constructs for recognition of TEV or Thrombin. Each peptide construct in the spot on the array comprises either a thrombin cleavage site or a TEV cleavage spot. The first surface of the assay tools comprises the specific peptide constructs, which are immobilized peptides that have dye-labeled 20-mer DNA sequences attached to them. This second surface of the assay tool has an immobilized 20-mer oligonucleotide (shown as small circles) complementary to the sequence attached to the peptide constructs adjacently placed in the second surface. The peptide constructs have dye-labeled 20-mer DNA sequences attached to them that are released upon cleavage of the peptide construct, which are complementary to the sequence attached to the reporter area. When a protease cleaves the peptide, the cleaved portion of the peptide containing labeled DNA diffuses towards the reporter area and hybridizes to its complementary oligonucleotide, leading to a fluorescent spot on the reporter area.

Solution containing TEV (Promega) or Thrombin (EMD Chemicals) proteases in a corresponding buffer was placed between the surface of the substrate slide with immobilized peptides and the surface of the reporter slide. The slide cartridge illustrated in FIG. 21 was used to assemble the GOS assay tool surface pairs. The assembled cartridge was incubated inside a humidified chamber at 30° C. After the reaction, the GOS array pair was disassembled, and the reporter slide was washed, dried, and scanned. A PE ScanArray Lite microarray reader with 5 μm resolution and 543 nm and 633 nm excitation was used to scan microscope slides with the peptide arrays.

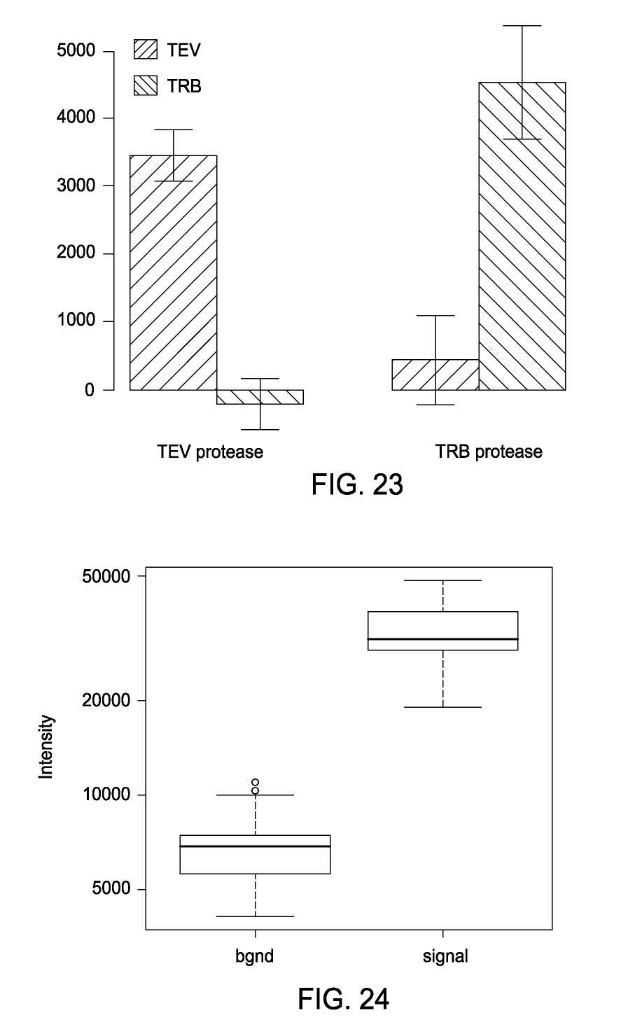

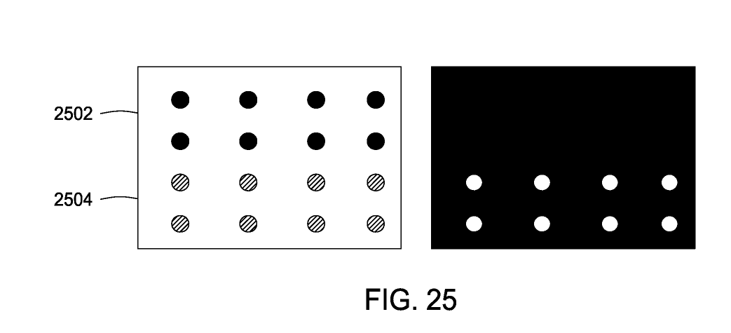

Strong signal from corresponding surfaces was achieved when the arrays were treated with TEV or thrombin proteases, while a very weak signal was present from the thrombin peptide substrate when the array was treated with TEV protease and vice versa (FIGS. 22 and 23). Similar weak levels of background signal were present when the arrays were treated with just buffer, omitting the proteases. This non-specific signal can be further reduced by using a non-protein blocking solution prior to peptide immobilization, choosing a peptide concentration that enables efficient immobilization but does not provide large excess of peptide resulting in non-covalent sticking to the slide, and/or establishing extensive wash procedures that ensure efficient removal of the unreacted peptides from the slide surface after immobilization. Such optimization techniques were able to reduce the background significantly, as evidenced in FIG. 24. The signal-to-noise ratio (S/N) of ˜28 achieved in the assay is over ten times higher than the S/N for traditional loss-of-signal assays as measured by loss of signal on the substrate slide, and a limit of detection comparable with techniques using individual peptide substrates (FIG. 25).

Reproducibility of the assay was assessed by treating 10 identical peptide arrays containing four spots for each of the TEV and thrombin protease substrates with the same solution of thrombin protease. intra- and inter-array signals were compared, and ANOVA analysis showed that array to array variation is highly significant (p<5.0e-07) and accounts for 76% of variation.

Dynamic Range and the Limit of Detection

The limit of detection (LOD) of the assay tool was determined by treating the TEV and thrombin protease substrate arrays with different concentrations of thrombin protease (FIG. 25). The LOD was determined as the minimum protease concentration at which signal corresponds to ≧3 times the standard deviation of the background. The calculated LOD is below 5.3 nM. The LOD for thrombin protease of the gain-of-signal protease activity assay is at least 4-fold better than colorimetric detection of thrombin by gold nanoparticles with attached aptamers capture (Pavlov V et al., J Am Chem. Soc. 2004 Sep. 29; 126(38):11768-9) and similar to that reported for single protease activity assays based on fluorescent conjugated polyelectrolytes (Pinto M R, Proc Natl Acad Sci USA. 2004 May 18; 101(20):7505-10. Epub 2004 May 10) or gold nanoparticles-based detection (Guarise C et al., Proc Natl Acad Sci USA. 2006 Mar. 14; 103(11):3978-82. Epub 2006 Mar. 1).

Comparison of Performance of the Gain-of-Signal Assay to a Loss-of-Signal Assay

The comparison of performance of the gain-of-signal (GOS) assay to a traditional loss of-signal (LOS) assay was done with our assay system by imaging both the slide comprising the capture agents and the substrate slides comprising the cleavable peptide constructs. The first substrate slide comprising the capture agent provided the data for the GOS assay, whereas the second substrate slide comprising the peptide constructs—substrates for both TEV (2502) and thrombin (2504) provided the data for the LOS assay. The substrate slide had to be scanned two times. The first scan was carried out before exposure to a protease containing sample in order to provide a baseline. The second scan was done after the treatment with proteases. If a peptide substrate is cleaved by a protease its intensity is expected to decrease in the second scan. The arrays were then treated with thrombin protease.

While both assays worked, the GOS assay produced more significant differences between signal and background. For the LOS assay, a paired t-test gave p<2.3e-03 for the comparison of before and after signals vs. p<2.2e-06 for the GOS assay. For the GOS assay, S/N=27.9 was calculated, while for the traditional LOS assay S/N=2.3. The S/N was defined as (signal gain)/stdev (background)=(mean reporter signal−mean background signal)/stdev(background), for the LOS assay the S/N was defined as (loss of signal)/stdev(signal before reaction). Therefore the data shown in FIG. 25 indicate that S/N for the new gain-of-signal (GOS) assay is more than 10 times higher compared to the traditional (LOS) assay.

Comparison of the GOS Assay to Activity Based Proteomics Profiling

Activity based proteomics profiling uses probes capable of covalently binding to is the active site of enzymes (Cravatt et al., Annu Rev Biochem. 2008; 77:383-414). This a very useful approach, but has some limitations: (1) large variations in protease structure limit the scope of small-molecule probes aimed at profiling entire class of enzymes; (2) low sensitivity is observed when the net quantity of proteases required to perform certain cellular tasks is low and occur in a background of high concentrations of inactive (e.g., zymogen or inhibitor bound) enzymes; (3) some proteases show poor cross-linking efficiency, a parameter that varies from enzyme to enzyme depending on the protein microenvironment surrounding the probe's reactive group; (4) active probes are suicide inhibitors, therefore the sensitivity is limited due to lack of enzymatic processivity. The assay tools and assays of the invention do not have these limitations, and thus allow screening of specific natural and unnatural protease peptide substrates for various applications including selection of probes for the activity based proteomics profiling technology.

Finally, the performance of the assay system was confirmed by determining the limit of detection (LOD) for Thrombin protease (<5.3 nM). The results found that the S/N of the new gain-of-signal assay was more than 10 fold higher than the S/N for conventional loss-of-signal assay formats.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the claims of the corresponding utility patents and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. §112, ¶6.

1. An assay tool for detecting biological or chemical activity in a sample, comprising:
a set of at least two different immobilized constructs, wherein the constructs comprises a releasable component; and
a capture surface; wherein the exposure of the immobilized constructs to a biological or chemical activity may result in release of a releasable component from one or more of the immobilized constructs, and wherein the interaction of a releasable component with the capture surface directly or indirectly enables the generation of a positive signal.

2. The assay tool of claim 1, wherein the positive signal identifies one or more immobilized constructs comprising a releasable component bound to the capture surface.

3. The assay tool of claim 1, wherein the capture surface comprises one or more capture agents for capture of the releasable component.

4. The assay tool of claim 3, wherein the releasable component comprises an affinity region that selectively binds to a capture agent on the capture surface.

5. The assay tool of claim 1, wherein the displacement event comprises enzymatic cleavage.

6. The assay tool of claim 5, wherein the enzymatic cleavage is cleavage by a protease.

7. An assay tool for detecting activity in a sample, comprising:
a set of at least two different immobilized constructs, wherein the constructs comprise a cleavage agent substrate region, an affinity region, and a detectable marker, wherein the affinity region and the detectable marker of a construct are released from the construct upon induced cleavage of the construct; and
a capture surface;
wherein binding of a released affinity region and detectable marker on a capture surface directly or indirectly enables the generation of a positive signal on the capture surface.

8. The tool of claim 7, wherein the constructs of the set comprise a substrate region for the same cleavage agent.

9. The tool of claim 7, wherein the constructs of the set comprise substrate regions for different cleavage agents.

10. An assay tool for detecting cleavage agent activity in a sample, comprising a surface having a set of two or more immobilized constructs comprising a cleavage agent substrate and a detectable marker;
and
a capture surface which produces a detectable positive signal upon cleavage of the substrate and binding of a released component comprising the detectable marker to the capture surface.

11. The tool of claim 10, wherein the capture surface comprises one or more capture agents that selectively bind to the released component.

12. The tool of claim 10, wherein the immobilized construct comprises an affinity region associated with the detectable marker, and wherein the capture agents selectively bind to the affinity region of the releasable component of the construct.

13. The tool of claim 10, wherein the capture agents on the capture surface are substantially identical.

14. The tool of claim 10, wherein the capture agents on the capture surface comprise two or more different capture agents.

15. The tool of claim 10, wherein two or more constructs of the set comprise different detectable markers.

16. The tool of claim 10, wherein the detectable markers of two or more constructs of the set are substantially identical.

17. The tool of claim 10, wherein the capture surface comprises a nucleic acid capture agent, and wherein the detectable marker is associated with a nucleic acid affinity region complementary to the capture agent.

18. The tool of claim 10, wherein the capture surface comprises a protein capture agent, and wherein the detectable marker is associated with a protein affinity region that specifically binds to the capture agent.

19. The tool of claim 9, wherein subsets of the constructs are physically separated on the surface.
